SEQUENCE LISTING (154 sequences)

SEQ ID NO: 1-4 - skipped (4 entries)

SEQ ID NO: 5 - protein, 26 aa, synthetic construct
  REGION 1..26: note = framework region 1
QVQLVESGGGLVQPGGSLRLSCAASG

SEQ ID NO: 6 - protein, 18 aa, synthetic construct
  REGION 1..18: note = framework region 2
MGWFRQAPGKEREFVAAI

SEQ ID NO: 7 - protein, 39 aa, synthetic construct
  REGION 1..39: note = framework region 3
YADSVKGRFTISRDNSKNTVYLQMNSLRPEDTAVYYCAA

SEQ ID NO: 8 - protein, 12 aa, synthetic construct
  REGION 1..12: note = framework region 4
YWGQGTLVTVSS

SEQ ID NO: 9 - protein, 7 aa, synthetic construct
  REGION 1..7: note = Nb42 CDR1
FAYSTYS

SEQ ID NO: 10 - protein, 8 aa, synthetic construct
  REGION 1..8: note = Nb42 CDR2
NSGTFRLW

SEQ ID NO: 11 - protein, 16 aa, synthetic construct
  REGION 1..16: note = Nb42 CDR3
RAWSPYSSTVDAGDFR

SEQ ID NO: 12-14 - skipped (3 entries)

SEQ ID NO: 15 - protein, 7 aa, synthetic construct
  REGION 1..7: note = aTNFa-mu CDR1
GTFSSII

SEQ ID NO: 16 - protein, 8 aa, synthetic construct
  REGION 1..8: note = aTNFa-mu CDR2
SWSGGTTV

SEQ ID NO: 17 - protein, 15 aa, synthetic construct
  REGION 1..15: note = aTNFa-mu CDR3
RPYQKYNWASASYNV

SEQ ID NO: 18 - protein, 7 aa, synthetic construct
  REGION 1..7: note = E1-1 CDR1
GGSDAGT

SEQ ID NO: 19 - protein, 8 aa, synthetic construct
  REGION 1..8: note = E1-1 CDR2
SWAGTAWR

SEQ ID NO: 20 - protein, 9 aa, synthetic construct
  REGION 1..9: note = E1-1 CDR3
LGSYEMDHH

SEQ ID NO: 21 - protein, 19 aa, synthetic construct
  REGION 1..19: note = aH linker
AEAAAKEAAAKEAAAKAGC

SEQ ID NO: 22 - protein, 21 aa, synthetic construct
  REGION 1..21: note = AE3K2R(2) linker
AEEEKRKAEEEKRKAEEEAGC

SEQ ID NO: 23 - protein, 28 aa, synthetic construct
  REGION 1..28: note = AE3K2R(3) linker
AEEEKRKAEEEKRKAEEEKRKAEEEAGC

SEQ ID NO: 24 - protein, 20 aa, synthetic construct
  REGION 1..20: note = E4K4(2) linker
AEEEEKKKKEEEEKKKKAGC

SEQ ID NO: 25 - protein, 14 aa, synthetic construct
  REGION 1..14: note = EA3K(2) linker
AEAAAKEAAAKAGC

SEQ ID NO: 26 - protein, 16 aa, synthetic construct
  REGION 1..16: note = Alfa linker
PSRLEEELRRRLTEGC

SEQ ID NO: 27 - protein, 77 aa, synthetic construct
  REGION 1..77: note = MyosinVI linker
AEEEEKKKQQEEEAERLRRIQEEMEKERKRREEDEERRRKEEEERRMKLEMEAKRKQEEEERKKREDDEKRKKKAGC

SEQ ID NO: 28 - protein, 13 aa, synthetic construct
  REGION 1..13: note = Spot linker
PDRVRAVSHWSSC

SEQ ID NO: 29 - protein, 20 aa, synthetic construct
  REGION 1..20: note = GT9 linker
GTGTGTGTGTGTGTGTGTGC

SEQ ID NO: 30 - protein, 17 aa, synthetic construct
  REGION 1..17: note = Modified Rigid linker
TPTTPPTPTPGTPPGGC

SEQ ID NO: 31-60 - skipped (30 entries)

SEQ ID NO: 61 - protein, 126 aa, synthetic construct
  REGION 1..126: note = Nb42
QVQLQESGGGSLQAGASLRLSCAASGFAYSTYSMGWFRQVSGKEREGVATINSGTFRLWYTDSVKGSFTISRDNAKNMLYLQMNSLKPEDTAIYYCAARAWSPYSSTVDAGDFRYWGQGTQVTVSS

SEQ ID NO: 62 - protein, 126 aa, synthetic construct
  REGION 1..126: note = HuNb42
QVQLVESGGGLVQPGGSLRLSCAASGFAYSTYSMGWFRQAPGKEREAVATINSGTFRLWYTDSVKGRFTISRDNSKNTLYLQMNSLRAEDTAVYYCAARAWSPYSSTVDAGDFRYWGQGTLVTVSS

SEQ ID NO: 63 - protein, 126 aa, synthetic construct
  REGION 1..126: note = HuNb42 P14A
QVQLVESGGGLVQAGGSLRLSCAASGFAYSTYSMGWFRQAPGKEREAVATINSGTFRLWYTDSVKGRFTISRDNSKNTLYLQMNSLRAEDTAVYYCAARAWSPYSSTVDAGDFRYWGQGTLVTVSS

SEQ ID NO: 64 - protein, 126 aa, synthetic construct
  REGION 1..126: note = HuNb42 T61A
QVQLVESGGGLVQPGGSLRLSCAASGFAYSTYSMGWFRQAPGKEREAVATINSGTFRLWYADSVKGRFTISRDNSKNTLYLQMNSLRAEDTAVYYCAARAWSPYSSTVDAGDFRYWGQGTLVTVSS

SEQ ID NO: 65 - protein, 126 aa, synthetic construct
  REGION 1..126: note = HuNb42 S75A
QVQLVESGGGLVQPGGSLRLSCAASGFAYSTYSMGWFRQAPGKEREAVATINSGTFRLWYTDSVKGRFTISRDNAKNTLYLQMNSLRAEDTAVYYCAARAWSPYSSTVDAGDFRYWGQGTLVTVSS

SEQ ID NO: 66 - protein, 126 aa, synthetic construct
  REGION 1..126: note = HuNb42 L79V
QVQLVESGGGLVQPGGSLRLSCAASGFAYSTYSMGWFRQAPGKEREAVATINSGTFRLWYTDSVKGRFTISRDNSKNTVYLQMNSLRAEDTAVYYCAARAWSPYSSTVDAGDFRYWGQGTLVTVSS

SEQ ID NO: 67 - protein, 126 aa, synthetic construct
  REGION 1..126: note = HuNb42 A88P
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTLVTVSS

SEQ ID NO: 68 - protein, 126 aa, synthetic construct
  REGION 1..126: note = HuNb42 L121Q
QVQLVESGGGLVQPGGSLRLSCAASGFAYSTYSMGWFRQAPGKEREAVATINSGTFRLWYTDSVKGRFTISRDNSKNTLYLQMNSLRAEDTAVYYCAARAWSPYSSTVDAGDFRYWGQGTQVTVSS

SEQ ID NO: 69 - protein, 126 aa, synthetic construct
  REGION 1..126: note = HuNb42 T61A A88P
QVQLVESGGGLVQPGGSLRLSCAASGFAYSTYSMGWFRQAPGKEREAVATINSGTFRLWYADSVKGRFTISRDNSKNTLYLQMNSLRPEDTAVYYCAARAWSPYSSTVDAGDFRYWGQGTLVTVSS

SEQ ID NO: 70 - protein, 126 aa, synthetic construct
  REGION 1..126: note = HuNb42 A88P L115Q
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTQVTVSS

SEQ ID NO: 71 - protein, 124 aa, synthetic construct
  REGION 1..124: note = aTNF-a mu
QVQLQDSGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTQVTVSS

SEQ ID NO: 72 - protein, 124 aa, synthetic construct
  REGION 1..124: note = aTNF-a mu 3MUT
QVQLQESGGGLVQAGGSLRLSCAASGGTFSSIIMAWFRQAPGKEREFVGAVSWSGGTTVYADSVKGRFTISRDSARKSVYLQMNSLKPEDTAVYYCAARPYQKYNWASASYNVWGQGTQVTVSS

SEQ ID NO: 73 - protein, 129 aa, synthetic construct
  REGION 1..129: note = aTNF-a VHH
QVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSS

SEQ ID NO: 74-80 - skipped (7 entries)

SEQ ID NO: 81 - protein, 119 aa, synthetic construct
  REGION 1..119: note = E1-1
EVQLQASGGGFVQPGGSLRLSCAASGGGSDAGTMGWFRQAPGKEREFVSAISWAGTAWRYYADSVKGRFTISRDNSKNTVYLQMNSLRAEDTATYYCALGSYEMDHHYWGQGTQVTVSS

SEQ ID NO: 82 - protein, 119 aa, synthetic construct
  REGION 1..119: note = E1-1 F11L
EVQLQASGGGLVQPGGSLRLSCAASGGGSDAGTMGWFRQAPGKEREFVSAISWAGTAWRYYADSVKGRFTISRDNSKNTVYLQMNSLRAEDTATYYCALGSYEMDHHYWGQGTQVTVSS

SEQ ID NO: 83 - protein, 119 aa, synthetic construct
  REGION 1..119: note = E1-1 S49A
EVQLQASGGGFVQPGGSLRLSCAASGGGSDAGTMGWFRQAPGKEREFVAAISWAGTAWRYYADSVKGRFTISRDNSKNTVYLQMNSLRAEDTATYYCALGSYEMDHHYWGQGTQVTVSS

SEQ ID NO: 84 - protein, 119 aa, synthetic construct
  REGION 1..119: note = E1-1 F11L S49A
EVQLQASGGGLVQPGGSLRLSCAASGGGSDAGTMGWFRQAPGKEREFVAAISWAGTAWRYYADSVKGRFTISRDNSKNTVYLQMNSLRAEDTATYYCALGSYEMDHHYWGQGTQVTVSS

SEQ ID NO: 85 - protein, 120 aa, synthetic construct
  REGION 1..120: note = E1-1 CDR
EVQLQASGGGFVQPGGSLRLSCAASGRRFSIEAMGWFRQAPGKEREFVSAIDSGGSTDYADSVKGRFTISRDNSKNTVYLQMNSLRAEDTATYYCAVIGSSWYGRGLDYWGQGTQVTVSS

SEQ ID NO: 86-90 - skipped (5 entries)

SEQ ID NO: 91 - protein, 125 aa, synthetic construct
  REGION 1..125: note = anti-VEGF VHH
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 92 - protein, 134 aa, synthetic construct
  REGION 1..134: note = anti-VEGF DARPIN
GSDLDKKLLEAARAGQDDEVRILMANGADVNARDSTGWTPLHLAAPWGHPEIVEVLLKNGADVNAADFQGWTPLHLAAAVGHLEIVEVLLKYGADVNAQDKFGKTAFDISIDNGNEDLAEILQKAAGGGSGGGS

SEQ ID NO: 93 - protein, 130 aa, synthetic construct
  REGION 1..130: note = anti-VEGF HuNb22 2MUT
QVQLVESGGGLVQPGGSLRLSCAASGYAYDTYYMGWFRQAPGKEREGVAGITSLVSGVAYYKYYTDSVKGRFTISRDNSKNTVDLQMNSLRAEDTAVYYCAASRSGLRARLLRPELYEYWGQGTLVTVSS

SEQ ID NO: 94 - protein, 127 aa, synthetic construct
  REGION 1..127: note = anti-VEGF HuNb23 3MUT
QVQLVESGGGLVQPGGSLRLSCVASGDTYSSACMGWFRQAPGKEREGVATICTSTSMRTRYYADSVKGRFTISRDNSKNTVYLQMNSLRAEDTAVYYCATGHTVGSSWRDPGAWRYWGQGTLVTVSS

SEQ ID NO: 95 - protein, 123 aa, synthetic construct
  REGION 1..123: note = anti-VEGF HuNb35 4MUT
QVQLVESGGGLVQPGGSLRLSCAASGLSYRPGYMGWFRQAPGKEREGVAIITTGGVTHYADSVKGRFTISRDNSKNTVYLQMNSLRAEDTAVYYCALANWVQFPLRVDGYKYWGQGTLVTVSS

SEQ ID NO: 96 - protein, 125 aa, synthetic construct
  REGION 1..125: note = Hu_aVEGF_VHH_3MUT
QVQLVESGGGLVQPGGSLRLSCAASGRTFSSYSMGWFRQAPGKEREFVAAISKGGYKYDAVSLEGRFTISRDNSKNTVYLQMNSLRPEDTAVYYCASSRAYGSSRLRLADTYEYWGQGTLVTVSS

SEQ ID NO: 97 - protein, 125 aa, synthetic construct
  REGION 1..125: note = Hu_aVEGF_VHH_5MUT
QVQLVESGGGLVQPGGSLRLSCAASGRTFSSYSMGWFRQAPGKEREFVAAISKGGYKYDAVSVKGRFTISRDNSKNTVYLQMNSLRPEDTAVYYCASSRAYGSSRLRLADTYEYWGQGTLVTVSS

SEQ ID NO: 98 - protein, 125 aa, synthetic construct
  REGION 1..125: note = Hu_aVEGF_VHH_6MUT
QVQLVESGGGLVQPGGSLRLSCAASGRTFSSYSMGWFRQAPGKEREFVAAISKGGYKYYAVSVKGRFTISRDNSKNTVYLQMNSLRPEDTAVYYCASSRAYGSSRLRLADTYEYWGQGTLVTVSS

SEQ ID NO: 99-100 - skipped (2 entries)

SEQ ID NO: 101 - protein, 150 aa, synthetic construct
  REGION 1..150: note = anti-TNFalpha VHH (human) - rigid
QVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSPSTPPTPSPSTPPGGCDDDDK

SEQ ID NO: 102 - protein, 148 aa, synthetic construct
  REGION 1..148: note = anti-TNFalpha VHH (human)- aH
QVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSAEAAAKEAAAKEAAAKAGC

SEQ ID NO: 103 - protein, 144 aa, synthetic construct
  REGION 1..144: note = anti-TNFalpha VHH (mouse)- aH
QVQLQDSGGGLVQAGGSLRLSCAASGGTFSSIIMAWFRQAPGKEREFVGAVSWSGGTTVYADSVLGRFEISRDSARKSVYLQMNSLKFPEDTAVYYCAARPYQKYNWASASYNVWGQGTQVTVSSAEAAAKEAAAKEAAAKAGC

SEQ ID NO: 104 - protein, 143 aa, synthetic construct
  REGION 1..143: note = anti-TNFalpha 3MUT VHH (mouse)- aH
QVQLQESGGGLVQAGGSLRLSCAASGGTFSSIIMAWFRQAPGKEREFVGAVSWSGGTTVYADSVKGRFTISRDSARKSVYLQMNSLKPEDTAVYYCAARPYQKYNWASASYNVWGQGTQVTVSSAEAAAKEAAAKEAAAKAGC

SEQ ID NO: 105 - protein, 62 aa, synthetic construct
  REGION 1..62: note = anti-TNFalpha affibody
CGGGVDNKFNKEVGWAFGEIGALPNLNALQFRAFIISLWDDPSQSANLLAEAKKLNDAQAPK

SEQ ID NO: 106 - protein, 265 aa, synthetic construct
  REGION 1..265: note = anti-IL-1beta scFv-rigid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSSSPSTPPTPSPSTPPGGC

SEQ ID NO: 107 - protein, 124 aa, synthetic construct
  REGION 1..124: note = Hu_aTNFaMu_3MUT
QVQLVESGGGLVQPGGSLRLSCAASGGTFSSIIMAWFRQAPGKEREFVGAVSWSGGTTVYADSVKGRFTISRDNSKNTVYLQMNSLRPEDTAVYYCAARPYQKYNWASASYNVWGQGTLVTVSS

SEQ ID NO: 108 - protein, 124 aa, synthetic construct
  REGION 1..124: note = Hu_aTNFaMu_5MUT
QVQLVESGGGLVQPGGSLRLSCAASGGTFSSIIMAWFRQAPGKEREFVAAISWSGGTTVYADSVKGRFTISRDNSKNTVYLQMNSLRPEDTAVYYCAARPYQKYNWASASYNVWGQGTLVTVSS

SEQ ID NO: 109 - protein, 129 aa, synthetic construct
  REGION 1..129: note = Hu_aTNFaHu_7MUT
QVQLVESGGGLVQPGGSLRLSCAASGRTFSDHSGYTYTMGWFRQAPGKEREFVARIYWSSGNTYYADSVKGRFTISRDNSKNTVYLQMNSLRPEDTAVYYCAARDGIPTSRSVESYNYWGQGTLVTVSS

SEQ ID NO: 110 - skipped

SEQ ID NO: 111 - protein, 124 aa, synthetic construct
  REGION 1..124: note = Hu_aEGFR_3MUT
EVQLVESGGGLVQPGGSLRLSCAASGRTSRSYGMGWFRQAPGKEREFVAGISWRGDSTGYADSVKGRFTISRDNSKNTVDLQMNSLRPEDTAVYYCAAAAGSAWYGTLYEYDYWGQGTLVTVSS

SEQ ID NO: 112 - protein, 120 aa, synthetic construct
  REGION 1..120: note = Hu_aHer2_3MUT
EVQLVESGGGLVQPGGSLRLSCAASGITFMRYAMGWYRQAPGKQREMVASINSGGTTNYADSVKGRFTISRDNSKNTVYLQMNSLRPEDTAVYYCNARWVKPQFIDNNYWGQGTLVTVSS

SEQ ID NO: 113 - protein, 119 aa, synthetic construct
  REGION 1..119: note = Hu_aPD1_102C3 3MUT
EVQLVESGGGLVQPGGSLRLSCAASGSIFSIHAMGWFRQAPGKEREFVAAITWSGGITYYEDSVKGRFTISRDNSKNTVYLQMNSLRPEDTAVYYCAADRAESSWYDYWGQGTLVTVSS

SEQ ID NO: 114 - protein, 119 aa, synthetic construct
  REGION 1..119: note = Hu_aPD1_102C12 3MUT
EVQLVESGGGLVQPGGSLRLSCAASGSIASIHAMGWFRQAPGKEREFVAVITWSGGITYYADSVKGRFTISRDNSKNTVYLQMNSLRPEDTAVYYCAGDKHQSSWYDYWGQGTLVTVSS

SEQ ID NO: 115 - protein, 119 aa, synthetic construct
  REGION 1..119: note = Hu_aPD1_102E2 3MUT
EVQLVESGGGLVQPGGSLRLSCAASGSISSIHAMGWFRQAPGKEREFVAAITWSGGITYYADSVKGRFTISRDNSKNTVYLQMNSLRPEDTAVYYCAADRAQSSWYDYWGQGTLVTVSS

SEQ ID NO: 116 - protein, 119 aa, synthetic construct
  REGION 1..119: note = Hu_aPD1_102E8 3MUT
EVQLVESGGGLVQPGGSLRLSCAASGSIFSINAMAWFRQAPGKEREFVALISWSGGSTYYEDSVKGRFTISRDNSKNTVYLQMNSLRPEDTAVYYCAADRVDSNWYDYWGQGTLVTVSS

SEQ ID NO: 117 - protein, 119 aa, synthetic construct
  REGION 1..119: note = Hu_aPD1_102H12 4MUT
EVQLVESGGGLVQPGGSLRLSCAASGRAFSSGTMGWFRQAPGKEREFVASIPWSGGRTYYADSVKGRFTISRDNSKNTVYLQMNSLRPEDTAVYYCAVKERSTGWDFAWGQGTLVTVSS

SEQ ID NO: 118 - protein, 119 aa, synthetic construct
  REGION 1..119: note = Hu aCaffeine 3MUT
EVQLVESGGGLVQPGGSLRLSCAASGRTGTIYSMAWFRQAPGKEREFLATIGWSSGITYYMDSVKGRFTISRDNSKNTVYLQMNSLRPEDTAVYYCAATRAYSVGYDYWGQGTLVTVSS

SEQ ID NO: 119-144 - skipped (26 entries)

SEQ ID NO: 145 - protein, 145 aa, synthetic construct
  REGION 1..145: note = HuNb42 A88P aH_CYS
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTLVTVSSAEAAAKEAAAKEAAAKAGC

SEQ ID NO: 146 - protein, 145 aa, synthetic construct
  REGION 1..145: note = HuNb42 T61A A88P aH_CYS
QVQLVESGGGLVQPGGSLRLSCAASGFAYSTYSMGWFRQAPGKEREAVATINSGTFRLWYADSVKGRFTISRDNSKNTLYLQMNSLRPEDTAVYYCAARAWSPYSSTVDAGDFRYWGQGTLVTVSSAEAAAKEAAAKEAAAKAGC

SEQ ID NO: 147 - protein, 145 aa, synthetic construct
  REGION 1..145: note = HuNb42 A88P L115Q aH_CYS
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTQVTVSSAEAAAKEAAAKEAAAKAGC

SEQ ID NO: 148 - protein, 65 aa, synthetic construct
  REGION 1..65: note = anti-TNFalpha affibody Gly
CGGGVDNKFNKEVGWAFGEIGALPNLNALQFRAFIISLWDDPSQSANLLAEAKKLNDAQAPKGGG

SEQ ID NO: 149 - protein, 143 aa, synthetic construct
  REGION 1..143: note = Hu_aTNFaMu_3MUT aH_CYS
QVQLVESGGGLVQPGGSLRLSCAASGGTFSSIIMAWFRQAPGKEREFVGAVSWSGGTTVYADSVKGRFTISRDNSKNTVYLQMNSLRPEDTAVYYCAARPYQKYNWASASYNVWGQGTLVTVSSAEAAAKEAAAKEAAAKAGC

SEQ ID NO: 150 - protein, 143 aa, synthetic construct
  REGION 1..143: note = Hu_aTNFaMu_5MUT aH_CYS
QVQLVESGGGLVQPGGSLRLSCAASGGTFSSIIMAWFRQAPGKEREFVAAISWSGGTTVYADSVKGRFTISRDNSKNTVYLQMNSLRPEDTAVYYCAARPYQKYNWASASYNVWGQGTLVTVSSAEAAAKEAAAKEAAAKAGC

SEQ ID NO: 151 - protein, 148 aa, synthetic construct
  REGION 1..148: note = Hu_aTNFaHu_7MUT aH_CYS
QVQLVESGGGLVQPGGSLRLSCAASGRTFSDHSGYTYTMGWFRQAPGKEREFVARIYWSSGNTYYADSVKGRFTISRDNSKNTVYLQMNSLRPEDTAVYYCAARDGIPTSRSVESYNYWGQGTLVTVSSAEAAAKEAAAKEAAAKAGC

SEQ ID NO: 152 - protein, 152 aa, synthetic construct
  REGION 1..152: note = IL-2_C125S aH_CYS
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTAEAAAKEAAAKEAAAKAGC

SEQ ID NO: 153 - protein, 133 aa, synthetic construct
  REGION 1..133: note = IL-15_5MUT aH_CYS
NWVNVISDLKKIEDLIQSMHIDATLYTESDVHPSCKVTAMQCFLSELQVISLESGDASIHDTVENLTILANNSLSSNGYVTESGCKECEELEAKNIKEFLQSFVHIVQMFINTSAEAAAKEAAAKEAAAKAGC

SEQ ID NO: 154 - protein, 166 aa, synthetic construct
  REGION 1..166: note = aTNFa_DARPin G3S_CYS
DLGKKLLEVARAGQDDEVRILMANGADVNAADHQSFTPLHLYAIFGHLEIVEVLLKNGADVNASDWHGNTPLHLAAWIGHLEIVEVLLKYGADVNATDHSGSTPLHLAATLGHLEIVEVLLKYGADVNAQDKFGKTAFDISIDNGNEDLAEILQKAAGGGSGGGSC

(19) United States
(12) Patent Application Publication
(10) Pub. No.: US 2025/0255972 A1
(43) Pub. Date: Aug. 14, 2025

(54) METHOD OF TREATING UVEITIS WITH MULTIVALENT PROTEIN-HYALURONIC ACID POLYMER CONJUGATE

(71) Applicant: Valitor, Inc., Berkeley, CA (US)

(72) Inventors: Wesley M. JACKSON, Berkeley, CA (US); Amy A TWITE, Berkeley, CA (US); Adam BARNEBEY, Berkeley, CA (US); Livia Wilz BRIER, Berkeley, CA (US)

(21) Appl. No.: 18/856,982

(22) PCT Filed: Apr. 14, 2023
(86) PCT No.: PCT/US2023/065779

§ 371 Date: Oct. 15, 2024

Related U.S. Application Data
(60) Provisional application No. 63/331,554, filed on Apr. 15, 2022.

(51) Int. Cl. A61K 47/61; A61K 39/00; A61K 47/36; A61P 35/00; C07K 16/24

(52) U.S. Cl. CPC A61K 47/61; A61K 47/36; A61P 35/00; C07K 16/241; A61K 2039/505; A61K 2039/545

(57) ABSTRACT

The present invention relates to multivalent protein-polymer conjugates, compositions, and methods for treating uveitis, such as chronic non-infectious uveitis.

### CROSS-REFERENCES TO RELATED APPLICATIONS

This application claims priority to U.S. Provisional Application No. 63/331,554, filed Apr. 15, 2022, which is incorporated herein in its entirety for all purposes.

### SEQUENCE LISTING

The material in the accompanying sequence listing is hereby incorporated by reference in its entirety. The accompanying file, named 2023-04-11 Sequence Listing_ST26 052566-508001WO.xml was created on Apr. 11, 2023, and is 117,702 bytes in size.

### BACKGROUND OF THE INVENTION

The use of biopolymers to modify the properties of biologically active agents is a recurring theme across a wide range of medical and biological applications. A variety of chemical linkers can be used to attach bioactive peptides or proteins to biopolymers to modify the pharmacological properties of the resulting conjugate for use as a drug that can provide optimal treatment of specific diseases. Peptide-polymer conjugates comprising multiple copies of one or more species of peptide conjugated to a single biopolymer chain have been employed to impart specific improvements to the pharmacological properties of the peptides, including: (1) higher binding affinity to the biological target, (2) slower diffusivity through a target tissue, and (3) inhibition of proteases that could deactivate the biological activity of the peptides or proteins.

These improved pharmacological properties of peptide-polymer conjugates are particularly useful for the delivery of potent drugs that are delivered directly into the diseased tissue. The dose delivered directly into the tissue can be lower than would be required to achieve the same therapeutic effect after systemic administration because the drug has been administered locally to the target tissue. It is also possible to administer to drugs to tissues that otherwise have poor transport properties from the blood. Specific examples of tissues where direct drug administration is common include the posterior eye chamber via intravitreal injection and articular joints via intra-articular injection.

However, local tissue administration requires a professional to safely provide the required injection, which makes them more burdensome and costly to administer compared to systemic administration. When the peptide drug is administered as part of a peptide-polymer conjugate, it is possible to substantially reduce the frequency of drug administration, thereby reducing the burden on the patient to receive effective treatment. Furthermore, a reduction in the number of local injections reduces the risk of local tissue injury or adverse effects to the injection. Finally, the need for less frequent administrations can reduce the amount of time that the drug concentration in the target tissue is below the therapeutic concentration, thereby improving the overall efficacy of the drug. Based on these advantages, there is a strong motivation to develop protein-polymer drug products for a variety of diseases.

Uveitis is a group of sight-threatening intraocular inflammation diseases that is responsible for roughly 5-10% of blindness cases worldwide. Chronic non-infectious uveitis can result in nerve damage and vision loss. Most patients are treated using corticosteroids, which can lead to serious side effects. Intravitreal administration of biologic TNFα inhibitors can substantially reduce the need for steroids. However, these products were not designed or validated for intravitreal use, and off-label intravitreal treatment with existing TNFα inhibitors is not recommended.

Therefore, there is a need to develop purified peptide-polymer conjugates and methods for treating uveitis, such as chronic non-infectious uveitis. The present invention meets this and other needs.

### BRIEF SUMMARY OF THE INVENTION

In some embodiments, the method of the present invention is a method for treating uveitis in a subject in need thereof, comprising administering to the subject a therapeutically effective amount of a conjugate of Formula V:

(X—Y)ₙ—Z (V),
wherein
each X is independently an anti-inflammatory peptide having a molecular weight of from about 5 kDa to about 200 kDa;
each Y is an organic linker;
Z is a hyaluronic acid polymer having a molecular weight of from about 0.1 MDa to about 3 MDa; and
subscript n is an integer of from 1 to 1000.

In some embodiments, the conjugate of the present invention is a random polymer of Formula VI:

(X—Y—Z¹)ₙ—(Z²)ₚ—(Z³)_{q} (VI),
having a molecular weight of from about 0.1 MDa to about 3 MDa;
wherein
each X is independently an anti-TNF-α or anti-IL-1β peptide comprising:

(SEQ ID NO: 101)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSPSTPPTPS PSTPPGGCDD
DDK,
(SEQ ID NO: 102)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSAEAAAKEA AAKEAAAKAG
C,
(SEQ ID NO: 103)
QVQLQDS GGGLVQAGGS LRLSCAASGG TFSSIIMAWF RQAPGKEREF
VGAVSWSGGT TVYADSVLGR FEISRDSARK SVYLQMNSLK PEDTAVYYCA
ARPYQKYNWA SASYNVWGQG TQVTVSSAEA AAKEAAAKEA AAKAGC,
(SEQ ID NO: 104)
QVQLQES GGGLVQAGGS LRLSCAASGG TFSSIIMAWF RQAPGKEREF
VGAVSWSGGT TVYADSVKGR FTISRDSARK SVYLQMNSLK PEDTAVYYCA
ARPYQKYNWA SASYNVWGQG TQVTVSSAEA AAKEAAAKEA AAKAGC,
(SEQ ID NO: 105)
CGGGVDNKFN KEVGWAFGEI GALPNLNALQ FRAFIISLWD DPSQSANLLA
EAKKLNDAQA PK,
or
(SEQ ID NO: 106)
EIVMTQS PSTLSASVGD RVIITCQASQ SIDNWLSWYQ QKPGKAPKLL
IYRASTLASG VPSRFSGSGS GAEFTLTISS LQPDDFATYY CQNTGGGVSI
AFGQGTKLTV LGGGGGSGGG GSGGGGSGGG GSEVQLVESG GGLVQPGGSL
RLSCTASGFS LSSAAMAWVR QAPGKGLEWV GIIYDSASTY YASWAKGRFT
ISRDTSKNTV YLQMNSLRAE DTAVYYCARE RAIFSGDFVL WGQGTLVTVS
SSPSTPPTPS PSTPPGGC;

each Y is an organic linker having the structure:

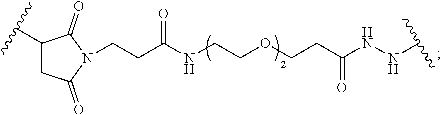

each X—Y—Z¹ moiety has the structure:

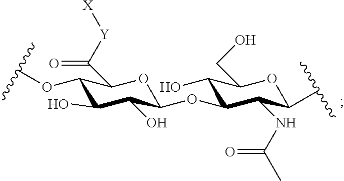

each Z² has the structure:

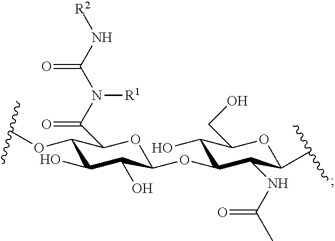

each Z³ independently has the structure:

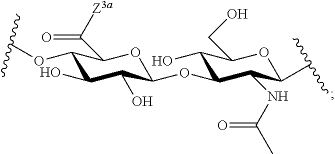

each R¹ and R² is independently C₁-C₆ alkyl, —(C₁-C₆ alkyl)-NR³R⁴, or C₅-C₈cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

### BRIEF DESCRIPTION OF THE DRAWINGS

FIG. 1 shows SDS-PAGE images of representative anti-inflammatory peptide polymer conjugates for each sequence ID. (A) SDS-PAGE image of (SEQ ID NO:101)+HyA (850 kDa) conjugate #2 with a valency of 55 compared to the unconjugated VHH. (B) SDS-PAGE image of (SEQ ID NO:102)+HyA (850 kDa) conjugate #3 with a valency of 65 compared to the unconjugated VHH. (C) SDS-PAGE image of (SEQ ID NO:103)+HyA (850 kDa) conjugate #5 with a valency of 121 compared to the unconjugated VHH. (D) SDS-PAGE image of (SEQ ID NO:104)+HyA (850 kDa) conjugate #6 with a valency of 51 compared to the unconjugated VHH. (E) SDS-PAGE image of (SEQ ID NO:105)+HyA (850 kDa) conjugate #8 with a valency of 21 compared to the unconjugated affibody. (F) SDS-PAGE image of (SEQ ID NO:106)+HyA (850 kDa) conjugate #9 with a valency of 15.

FIG. 2 shows (A) the TNFα binding affinity of a (SEQ ID NO:102)+HyA (850 kDa) conjugate #4 with a valency of 120 is greater than that of an unconjugated TNFα, as determined by biolayer interferometry (*** p<0.001 Student's t-tests with n=3) Dashed line indicates the limit of detection for the instrument and the binding affinity of the conjugate is below that limit. (B) The bioactivity of a (SEQ ID NO: 101)+HyA (850 kDa) conjugate #1 with a valency of 9 to inhibit TNFα-induced apoptosis in L929 fibroblasts is ˜10 fold greater than the unconjugated VHH.

FIG. 3 shows the hydrodynamic radius of conjugate #3 consisting of (SEQ ID NO:102)+HyA (850 kDa) with a valency of 65 is greater than that of an unconjugated VHH. (*** p<0.001 Student's t-tests with n=3).

FIG. 4 shows (A) the normalized absorbance at 280 nm (“A280”) in unconjugated protein SEQ ID NO:102 as the temperature increased. The oxidized version of the VHH showed minimal change in absorbance when the temperature increased from 37° C.-50° C. whereas the reduced construct showed increased absorbance starting at 50° C., indicating that it has unfolded and was less thermally stable. Error bars represent SD; (B) change in association constant of the TNFα binding affinity to mu_anti-TNFα_aH_CYS conjugates with or without the 3Mut stability enhancement mutation after incubation at 37° C. for the indicated number of days as determined by biolayer interferometry. The samples used were either (SEQ ID NO:103)+HyA (850 kDa) conjugate #5 with a valency of 121 (“mu anti-TNFα_aH_CYS MVP”) or (SEQ ID NO:104)+HyA (850 kDa) conjugate #6 with a valency of 51 (“mu anti-TNFα_3Mut_aH_CYS MVP”). After 4 days at 37° C., the association constant of conjugate #6 had minimal change after 35 days at 37° C. In contrast, the association constant of the non-mutated conjugate #5 dramatically decreased after 5 days at 37° C., indicative of decreased stability; (C) representative DLS data conjugate size after incubation at 37° C. in vitreous mimetic buffer for up to 35 days. Conjugates were made with (SEQ ID NO:104)+HyA (850 kDa) with a valency of 51 (conjugate #6, “mu_anti-TNFα_3Mut_aH_CYS MVP 1:51”) or 98 (conjugate #7, “mu_anti-TNFα_3Mut_aH_CYS MVP 1:98”). There was no significant difference in MVP size based on valency range from ˜50 to ˜100 antibodies per polymer. The conjugates slowly decreased in size to about 75% of the original radius after 35 days at 37° C. Error bars represent SD.

FIG. 5 shows that conjugation can increase the intravitreal half-life of an anti-inflammatory therapeutic in rabbit intravitreal pharmacokinetics model. Each rabbit received an equal molar 50 μL intravitreal injection of either unconjugated SEQ ID NO:102 or (SEQ ID NO:102)+HyA (850 kDa) conjugate #4 with a valency of 120. The intravitreal half-life was determined using a non-linear fit of the VHH concentration at each timepoint. Multivalent conjugation increased the half-life at least 2× compared to unconjugated VHH.

FIG. 6 shows that an anti-TNFα conjugate sufficiently suppressed ocular inflammation in a rat experimental autoimmune uveoretinitis model. Conjugate #7 was made with (SEQ ID NO:104)+HyA (850 kDa) and a valency of 98. (A) Average inflammation score (0=none to 4=severe) observed in rat eyes after EAU induction in rats injected intravitreally with either vehicle, dexamethasone (5 μg) or Conjugate #7 (“mu_anti-TNFα_3Mut_aH_CYS MVP”) (12.5 μg) (n=8). Both dexamethasone and conjugate treated eyes showed decreased inflammation compared to the vehicle control one day after ITV treatment. (B) Average histology inflammation scores (0=none to 4=severe) in the same cohort of rats that were sacrificed 14 days after model induction. Left bar: vehicle, middle bar: dexamethasone treated, right bar: Conjugate #7 treated. The conjugate treated eyes (right bar) were less inflamed than vehicle treated eyes (left bar) (* p<0.05 Student's t-tests).

FIG. 7A-7B show that an anti-TNFα conjugate sufficiently suppressed ocular inflammation in a rat experimental autoimmune uveoretinitis model. Conjugate #10 was made with (SEQ ID NO:104)+HyA (850 kDa) and a valency of 96.5. (A) Average inflammation score (0=none to 4=severe) observed in rat eyes after EAU induction in rats injected intravitreally with either vehicle (n=24), triamcinolone (40 μg) (n=22) or conjugate #10 (“Conjugate ID: 10”) (19 g) (n=20). Both triamcinolone and conjugate treated eyes showed decreased inflammation compared to the vehicle control one day after ITV treatment. No statistical difference was measured between triamcinolone and conjugate #10 at any timepoint. Conjugate #10 and vehicle were significantly different at Day 10, p=0.002, at day 12, p<0.001 and at day 14, p=0.007. (B) Cytokine analysis of vitreous samples prepared from rat eyes injected intravitreally with either vehicle (n=24), triamcinolone (40 μg) (n=22) or conjugate #10 (“Conjugate ID: 10”) (19 μg) (n=20). Bar graphs indicate the concentration of key pro-inflammatory cytokines and inflammatory regulators.

FIGS. 8A-8B show that an anti-TNFα conjugate sufficiently suppressed ocular inflammation in a rabbit TNFα-induced ocular inflammation model. Conjugate #11 was made with (SEQ ID NO:102)+HyA (850 kDa) and a valency of 132. (A) Average inflammation score over time (O-none to 4=severe) observed in rabbit eyes after inflammation induction using 7.5 μg of TNFα in rabbits injected intravitreally with either vehicle, or conjugate #11 (“Conjugate ID 11”) (0.26 mg) or no injection (n=26). In the rabbits treated with 7.5 μg of TNFα, there was a statistical significance between conjugate #11 and vehicle treated eyes at 24 and 48 hours. (B) After 48 hours, at all concentrations of TNFα dosed, conjugate #11 treated eyes showed decreased inflammation compared to the vehicle control 48 hours after inflammation induction. A statistically significant difference was observed at 5.0 μg (p=0.03) and 7.5 μg (p<0.001) of TNFα dosed.

### DETAILED DESCRIPTION OF THE INVENTION

### I. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Alkyl” is a linear or branched saturated monovalent or divalent hydrocarbon. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., (C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g. tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

“Organic linker” as used herein refers to a chemical moiety that directly or indirectly covalently links the peptide to the polymer. Organic linkers useful in the present invention can be about 100 Da to 500 Da. The types of organic linkers of the present invention include, but are not limited to, imides, amides, amines, esters, carbamates, ureas, thioethers, thiocarbamates, thiocarbonate and thioureas. One of skill in the art will appreciate that other types of organic linkers are useful in the present invention.

“Thiol” refers to the —SH functional group.

“Thiol reactive group” refers to a group capable of reacting with a thiol to form a covalent bond to the sulfur atom. Representative thiol reactive groups include, but are not limited to, thiol, TNB-thiol, haloacetyl, aziridine, acryloyl, vinylsulfone, APN (3-arylpropiolonitrile), maleimide and pyridyl disulfide. Reaction of the thiol reactive group with a thiol can form a disulfide or a thioether.

“Peptide,” “polypeptide,” and “protein” are used interchangeably herein, and refer to naturally occurring and synthetic amino acids of any length, as well as amino acid analogs and amino acid mimetics that function in a manner similar to the naturally occurring amino acids. The term “peptide” includes fusion proteins, including, but not limited to, fusion proteins with a heterologous amino acid sequence, fusions with heterologous and homologous leader sequences, with or without N-terminal methionine residues; immunologically tagged proteins; and the like. Peptides further include post-translationally modified peptides.

“VHH” as used herein refers to a single-domain heavy chain antibody.

“DARPin” refers to a designed ankyrin repeat protein, which is a genetically engineered antibody mimetic protein that can exhibit highly specific and high-affinity target protein binding.

An “alpha-helix” or “a-helix” is a common motif in the secondary structure of proteins and is a right hand-helix conformation in which every backbone N—H group hydrogen bonds to the backbone C═O group of the amino acid located four residues earlier along the protein sequence. The alpha-helix is also known as a classic Pauling-Corey-Branson α-helix, or 3.6₁₃-helix, which denotes the average number of residues per helical turn (3.6) with 13 atoms being involved in the ring formed by the hydrogen bond. Peptides that contain an alpha-helix is said to be alpha-helical. Such peptides may be partly or entirely alpha-helical. As understood in the art, an alpha-helix has at least four amino acid residues. In some embodiments, an alpha-helix has from 4 to 40 amino acids.

Provided are also pharmaceutically acceptable salts of the compounds or peptides described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

“Pharmaceutical composition” as used herein refers to a product comprising the specified ingredients in the specified amounts, as well as any product, which results, directly or indirectly, from combination of the specified ingredients in the specified amounts. The pharmaceutical composition is generally safe for biological use.

“Pharmaceutically acceptable excipient” as used herein refers to a substance that aids the administration of an active agent to an absorption by a subject. Pharmaceutically acceptable excipients useful in the present invention include, but are not limited to, binders, fillers, disintegrants, lubricants, coatings, sweeteners, flavors and colors. One of skill in the art will recognize that other pharmaceutically acceptable excipients are useful in the present invention.

The conjugates described herein may be prepared and/or formulated as pharmaceutically acceptable salts or when appropriate as a free base. Pharmaceutically acceptable salts are non-toxic salts of a free base form of a compound that possess the desired pharmacological activity of the free base. These salts may be derived from inorganic or organic acids or bases. For example, a conjugate that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, y-hydroxybutyrates, glycolates, tartrates, and mandelates. Lists of other suitable pharmaceutically acceptable salts are found in Remington: The Science and Practice of Pharmacy, 21^{st} Edition, Lippincott Williams and Wilkins, Philadelphia, Pa., 2006.

Examples of “pharmaceutically acceptable salts” of the conjugates disclosed herein also include salts derived from an appropriate base, such as an alkali metal (for example, sodium, potassium), an alkaline earth metal (for example, magnesium), ammonium and NR₄+(wherein R is C₁-C₄ alkyl). Also included are base addition salts, such as sodium or potassium salts.

“Therapeutically effective amount” as used herein refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can be lower than the conventional therapeutically effective dose for non-sensitized cells.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Prophylaxis” refers to preventing or retarding the progression of clinical illness in patients suffering from a disease.

A “subject” of the present invention is a mammal, which can be a human or a non-human mammal, for example a companion animal, such as a dog, cat, rat, or the like, or a farm animal, such as a horse, donkey, mule, goat, sheep, pig, or cow, and the like. In some embodiments, the subject is human.

### II. Conjugates

In some embodiments, the conjugate of the present invention is a conjugate of Formula V:

(X—Y)ₙ—Z (V),
wherein
   each X is independently an anti-inflammatory peptide having a molecular weight of from about 5 kDa to about 200 kDa;
   each Y is an organic linker;
   Z is a hyaluronic acid polymer having a molecular weight of from about 0.1 MDa to about 3 MDa; and
subscript n is an integer of from 1 to 1000.

In some embodiments, each X is independently an anti-TNF-α peptide or an anti-interleukin-1β peptide.

In some embodiments, each X is a monoclonal IgG, an IgG fragment, single chain scFv, single-domain heavy-chain VHH, adnectin, affibody, anticalin, DARPin, or an engineered Kunitz-type inhibitor. In some embodiments, each X is a monoclonal IgG. In some embodiments, each X is an IgG fragment. In some embodiments, each X is a single-domain heavy-chain VHH. In some embodiments, each X is a DARPin.

In some embodiments, each X is a peptide having an amino acid sequence comprising any one of SEQ ID NOS: 61-73, 81-85, 91-98, 101-109, 111-118, and 145-151. In some embodiments, each X is a peptide having an amino acid sequence comprising any one of SEQ ID NOS: 101-109 and 148-154.

In some embodiments, each X is a peptide having an amino acid sequence comprising any one of SEQ ID NOS: 61-73, 81-85, 91-95, 101-106, and 111-118.

In some embodiments, each X is a peptide having an amino acid sequence comprising:

(SEQ ID NO: 101)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSPSTPPTPS PSTPPGGCDD
DDK,
(SEQ ID NO: 102)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSAEAAAKEA AAKEAAAKAG
C,
(SEQ ID NO: 103)
QVQLQDS GGGLVQAGGS LRLSCAASGG TFSSIIMAWF RQAPGKEREF
VGAVSWSGGT TVYADSVLGR FEISRDSARK SVYLQMNSLK PEDTAVYYCA
ARPYQKYNWA SASYNVWGQG TQVTVSSAEA AAKEAAAKEA AAKAGC,
(SEQ ID NO: 104)
QVQLQES GGGLVQAGGS LRLSCAASGG TFSSIIMAWF RQAPGKEREF
VGAVSWSGGT TVYADSVKGR FTISRDSARK SVYLQMNSLK PEDTAVYYCA
ARPYQKYNWA SASYNVWGQG TQVTVSSAEA AAKEAAAKEA AAKAGC,
(SEQ ID NO: 105)
CGGGVDNKFN KEVGWAFGEI GALPNLNALQ FRAFIISLWD DPSQSANLLA
EAKKLNDAQA PK,
or
(SEQ ID NO: 106)
EIVMTQS PSTLSASVGD RVIITCQASQ SIDNWLSWYQ QKPGKAPKLL
IYRASTLASG VPSRFSGSGS GAEFTLTISS LQPDDFATYY CQNTGGGVSI
AFGQGTKLTV LGGGGGSGGG GSGGGGSGGG GSEVQLVESG GGLVQPGGSL
RLSCTASGFS LSSAAMAWVR QAPGKGLEWV GIIYDSASTY YASWAKGRFT
ISRDTSKNTV YLQMNSLRAE DTAVYYCARE RAIFSGDFVL WGQGTLVTVS
SSPSTPPTPS PSTPPGGC.

In some embodiments, each X is a peptide having an amino acid sequence comprising SEQ ID NO: 101. In some embodiments, each X is a peptide having an amino acid sequence comprising SEQ ID NO: 102. In some embodiments, each X is a peptide having an amino acid sequence comprising SEQ ID NO: 103. In some embodiments, each X is a peptide having an amino acid sequence comprising SEQ ID NO: 104. In some embodiments, each X is a peptide having an amino acid sequence comprising SEQ ID NO: 105. In some embodiments, each X is a peptide having an amino acid sequence comprising SEQ ID NO: 106.

Each peptide can be linked to the biocompatible polymer by a variety of organic linkers generally known in the art for forming antibody-drug conjugates, such as those provided by BroadPharm of San Diego, CA. Methods for forming bioconjugate bonds are described in Bioconjugate Techniques, 3^{rd} Edition, Greg T. Hermanson. The organic linkers can be reactive with amines, carbonyls, carboxyl and activated esters, can react via Click-chemistry (with or without copper), or be reactive with thiols.

Representative organic linkers include an amide or disulfide, or are formed from a reactive group such as succinic anhydride, succinimide, N-hydroxy succinimide, N-chlorosuccinimide, N-bromosuccinimide, maleic anhydride, maleimide, hydantoin, phthalimide, and others. The organic linkers useful in the present invention are small and generally have a molecular weight from about 100 Da to about 500 Da containing two functional groups consisting of a maleimide and either an amine or hydrazide. In some embodiments, the peptide is covalently linked to the polymer via a sulfide bond and an organic linker having a molecular weight of from about 100 Da to about 500 Da. In some embodiments, the organic linker has a molecular weight of from about 100 Da to about 300 Da. In some embodiments, the organic linker comprises a succinimide. In some embodiments, the organic linker is formed using N-beta-maleimidopropionic acid hydrazide (BMPH), N-epsilon-maleimidocaproic acid hydrazide (EMCH), N-aminoethylmaleimide, N-kappa-maleimidoundecanoic acid hydrazide (KUMH), hydrazide-PEG2-maleimide, amine-PEG2-maleimide, hydrazide-PEG3-maleimide, or amine-PEG3-maleimide.

In some embodiments, the organic linker has the structure:

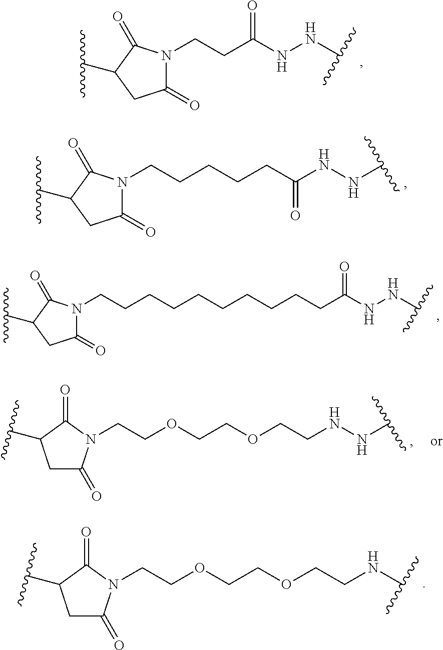

In some embodiments, the organic linker can be N-epsilon-maleimidocaproic acid hydrazide (EMCH):

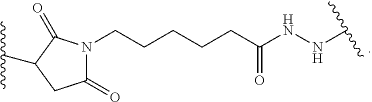 [SMILES: *NNC(=O)CCCCCN1C(=O)CC(*)C1=O]

In some embodiments, the organic linker has the structure:

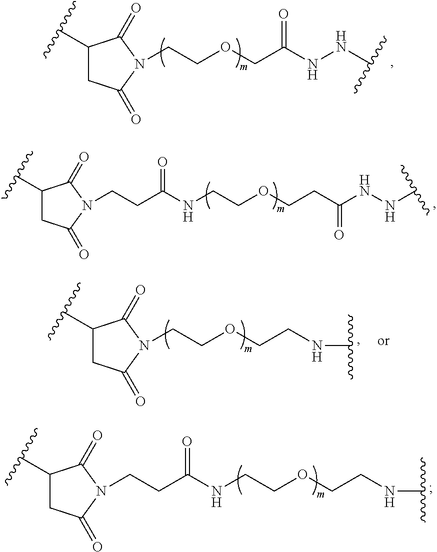

subscript m is an integer from 1 to 300. In some embodiments, subscript m is an integer from 1 to 100.

In some embodiments, the organic linker has the structure:

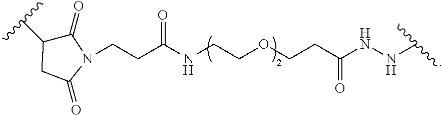

The organic linker with the above structure is known as MP2H.

In some embodiments, each Y is an organic linker having the structure:

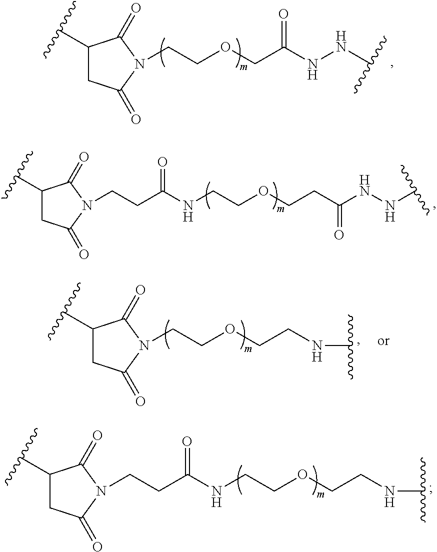

and
subscript m is an integer of from 1 to 300.

In some embodiments, Z has a molecular weight of from about 0.4 MDa to about 2 MDa. In some embodiments, Z has a molecular weight of from about 0.7 MDa to about 1.5 MDa. In some embodiments, Z has a molecular weight of about 0.8 MDa.

In some embodiments, the conjugate of Formula V has the structure of Formula Va:

(X¹—X²—Y)ₙ—Z (Va),
wherein
each X¹ is an anti-TNF-α peptide or an anti-interleukin-1β peptide having a molecular weight of from about 5 kDa to about 200 kDa;
each X² is a peptide linker that comprises an alpha-helix;
each Y is an organic linker having the structure:

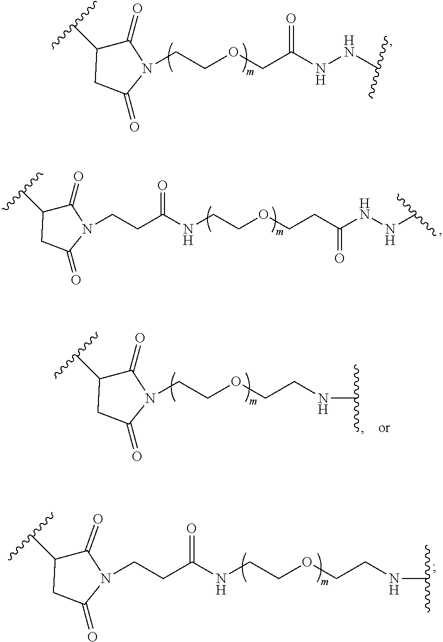

Z is a hyaluronic acid polymer having a molecular weight of from about 0.1 MDa to about 3 MDa; and
subscript m is an integer of from 1 to 300.

In some embodiments, each X¹ is a peptide having an amino acid sequence comprising any one of SEQ ID NOS: 61-73, 81-85, 91-98, 101-109, and 111-118.

In some embodiments, each X² is a peptide linker having an amino acid sequence comprising:

(SEQ ID NO: 21)
AEAAAKEAAAKEAAAKAGC,
(SEQ ID NO: 22)
AEEEKRKAEEEKRKAEEEAGC,
(SEQ ID NO: 23)
AEEEKRKAEEEKRKAEEEKRKAEEEAGC,
(SEQ ID NO: 24)
AEEEEKKKKEEEEKKKKAGC,
(SEQ ID NO: 25)
AEAAAKEAAAKAGC,
(SEQ ID NO: 26)
PSRLEEELRRRLTEGC,
or
(SEQ ID NO: 27)
AEEEEKKKQQEEEAERLRRIQEEMEKERKRREEDEERRRKEEEER
RMKLEMEAKRKQEEEERKKREDDEKRKKKAGC.

In some embodiments, each X² is a peptide linker having an amino acid sequence comprising AEAAAKEAAAKEAAAKAGC (SEQ ID NO: 21).

In some embodiments, each X¹ is a peptide having an amino acid sequence comprising SEQ ID NO: 107, and each X² is a peptide linker having an amino acid sequence comprising SEQ ID NO: 21. In some embodiments, each X¹ is a peptide having an amino acid sequence comprising SEQ ID NO: 108, and each X² is a peptide linker having an amino acid sequence comprising SEQ ID NO: 21. In some embodiments, each X¹ is a peptide having an amino acid sequence comprising SEQ ID NO: 109, and each X² is a peptide linker having an amino acid sequence comprising SEQ ID NO: 21.

In some embodiments, the conjugate of Formula V is a random polymer of Formula VI:

(X—Y—Z¹)ₙ—(Z²)ₚ—(Z³)_{q} (VI),
having a molecular weight of from about 0.1 MDa to about 3 MDa;
wherein
each X is independently an anti-inflammatory peptide having a molecular weight of from about 5 kDa to about 200 kDa;
each Y is an organic linker;
each X—Y—Z¹ moiety has the structure:

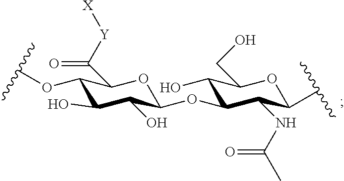

each Z² has the structure:

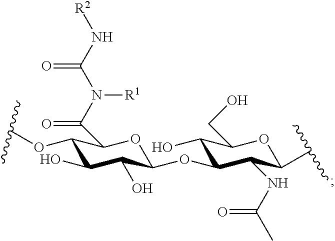

each Z³ independently has the structure:

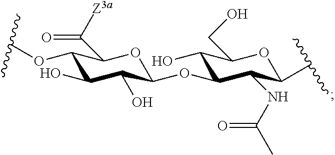

each R¹ and R² is independently C₁-C₆ alkyl, —(C₁-C₆ alkyl)-NR³R⁴, or C₅-C₈cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

In some embodiments, each X is a peptide having an amino acid sequence comprising any one of SEQ ID NOS: 61-73, 81-85, 91-98, 101-109, 111-118, and 145-154. In some embodiments, each X is a peptide having an amino acid sequence comprising any one of SEQ ID NOS: 101-109 and 148-154.

In some embodiments, each R¹ and R² is independently C₁-C₃ alkyl or —(C₁-C₃ alkyl)-NR³R⁴. In some embodiments, each R¹ and R² is ethyl or —(CH₂)₃—NMe₂. In some embodiments, each R¹ is ethyl; and each R² is —(CH₂)₃—NMe₂. In some embodiments, each R¹ is (CH₂)₃—NMe₂; and each R² is ethyl.

In some embodiments, each R³ and R⁴ is independently C₁-C₃ alkyl.

In some embodiments, preparing the conjugates of the present invention comprises covalently attaching the organic linker to the biocompatible polymer and then covalently attaching the peptide to the organic linker. In some embodiments, after preparing the conjugate of the present invention, unreacted organic linker is present on the biocompatible polymer. The structure of the unreacted organic linker depends on the organic linker and would be understood by a person skilled in the art.

Representative unreacted organic linkers include, but are not limited to,

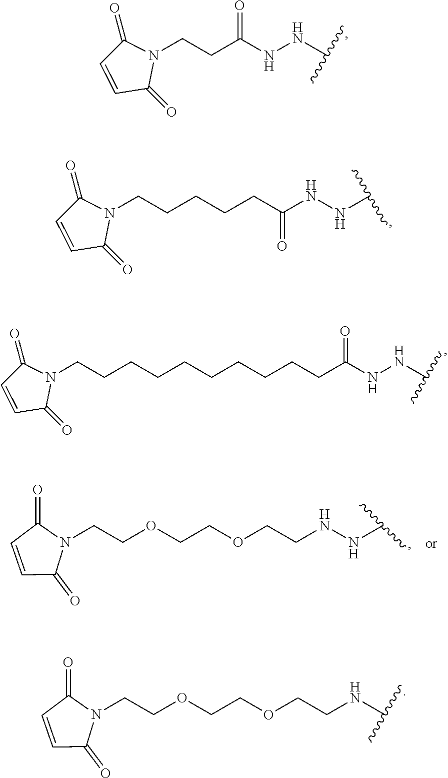 [SMILES: *NNC(=O)CCN1C(=O)C=CC1=O ; *NNC(=O)CCCCCN1C(=O)C=CC1=O ; *NNC(=O)CCCCCCCCCCN1C(=O)C=CC1=O ; *NNCCOCCOCCN1C(=O)C=CC1=O ; *NCCOCCOCCN1C(=O)C=CC1=O]

In some embodiments, the unreacted organic linker has the structure:

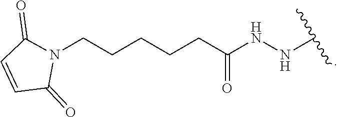 [SMILES: *NNC(=O)CCCCCN1C(=O)C=CC1=O]

In some embodiments, the unreacted organic linker has the structure:

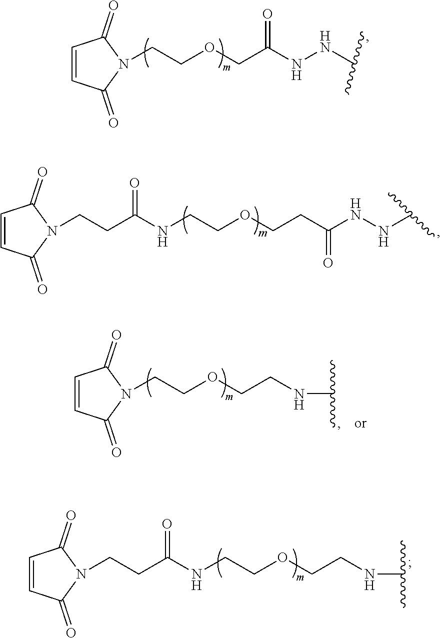

wherein subscript m is an integer of from 1 to 300. In some embodiments, subscript m is an integer from 1 to 100.

In some embodiments, the unreacted organic linker has the structure:

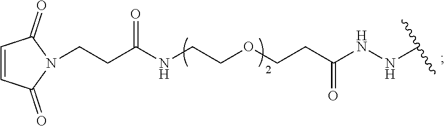

In some embodiments, the organic linker has the structure:

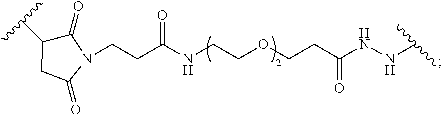

and
the unreacted organic linker has the structure:

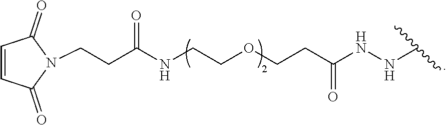

In some embodiments, the conjugate is a random polymer of Formula VI:

(X—Y—Z¹)ₙ—(Z²)ₚ—(Z³)_{q} (VI),
having a molecular weight of from about 0.1 MDa to about 3 MDa;
wherein
each X is independently an anti-TNF-α or anti-IL-1β peptide comprising:

(SEQ ID NO: 101)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSPSTPPTPS PSTPPGGCDD
DDK,
(SEQ ID NO: 102)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSAEAAAKEA AAKEAAAKAG
C,
(SEQ ID NO: 103)
QVQLQDS GGGLVQAGGS LRLSCAASGG TFSSIIMAWF RQAPGKEREF
VGAVSWSGGT TVYADSVLGR FEISRDSARK SVYLQMNSLK PEDTAVYYCA
ARPYQKYNWA SASYNVWGQG TQVTVSSAEA AAKEAAAKEA AAKAGC,
(SEQ ID NO: 104)
QVQLQES GGGLVQAGGS LRLSCAASGG TFSSIIMAWF RQAPGKEREF
VGAVSWSGGT TVYADSVKGR FTISRDSARK SVYLQMNSLK PEDTAVYYCA
ARPYQKYNWA SASYNVWGQG TQVTVSSAEA AAKEAAAKEA AAKAGC,
(SEQ ID NO: 105)
CGGGVDNKFN KEVGWAFGEI GALPNLNALQ FRAFIISLWD DPSQSANLLA
EAKKLNDAQA PK,
or
(SEQ ID NO: 106)
EIVMTQS PSTLSASVGD RVIITCQASQ SIDNWLSWYQ QKPGKAPKLL
IYRASTLASG VPSRFSGSGS GAEFTLTISS LQPDDFATYY CQNTGGGVSI
AFGQGTKLTV LGGGGGSGGG GSGGGGSGGG GSEVQLVESG GGLVQPGGSL
RLSCTASGFS LSSAAMAWVR QAPGKGLEWV GIIYDSASTY YASWAKGRFT
ISRDTSKNTV YLQMNSLRAE DTAVYYCARE RAIFSGDFVL WGQGTLVTVS
SSPSTPPTPS PSTPPGGC;

each Y is an organic linker having the structure:

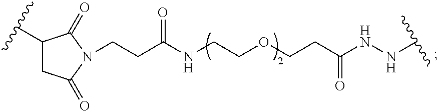

each X—Y—Z¹ moiety has the structure:

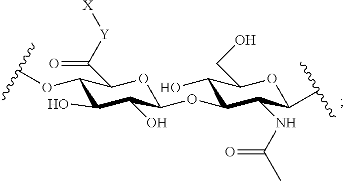

each Z² has the structure:

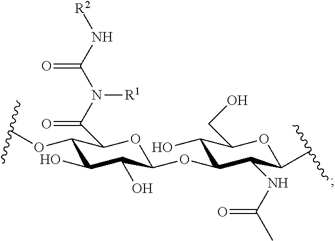

each Z³ independently has the structure:

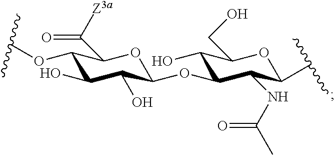

each R¹ and R² is independently C₁-C₆ alkyl, —(C₁-C₆ alkyl)-NR³R⁴, or C₅-C₈cycloalkyl;
each R³ and R⁴ is independently H or C₁-C₆ alkyl;
each Z³a is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

In some embodiments, subscript n is an integer of from 1 to 1500 and less than about 15% of the sum of subscripts n, p, and q; subscript p is an integer of from 1 to 1000 and less than about 10% of the sum of subscripts n, p, and q; and subscript q is an integer of from 100 to 10000. In some embodiments, subscript n is an integer of from 1 to 1000 and less than about 10% of the sum of subscripts n, p, and q; subscript p is an integer of from 1 to 800 and less than about 8% of the sum of subscripts n, p, and q; and subscript q is an integer of from 100 to 10000. In some embodiments, subscript n is an integer of from 10 to 450 and less than about 15% of the sum of subscripts n, p, and q; subscript p is an integer of from 1 to 300 and less than about 10% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 240 and less than about 8% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 60 and less than about 2% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 30 and less than about 1% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 15 and less than about 0.5% of the sum of subscripts n, p, and q; and subscript q is an integer of from 1000 to 3000.

In some embodiments, a conjugate of the present invention is for use in a method of treating uveitis as described herein.

### III. Compositions

In some embodiments, the present invention relates to a pharmaceutical composition as described herein. In some embodiments, the pharmaceutical composition is a pharmaceutical composition comprising a conjugate as described herein, and a pharmaceutically acceptable excipient.

### A. Formulation

For preparing pharmaceutical compositions from the conjugates of the present invention, pharmaceutically acceptable carriers can be either solid or liquid. Solid form preparations include powders, cachets, and dispersible granules. A solid carrier can be one or more substances, which may also act as diluents, binders, preservatives, disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the conjugates of the present invention.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the conjugates of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolality.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the conjugates of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration into a body cavity such as intravitreal administration into an eye or the intra-articular space of a joint. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV or intravitreal administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

### B. Administration

The conjugates and compositions of the present invention can be delivered by any suitable means, including oral, parenteral and topical methods. In some embodiments, the delivery method is intravitreal.

The pharmaceutical preparation is preferably in unit dosage form. In such form the preparation is subdivided into unit doses containing appropriate quantities of the conjugates and compositions of the present invention. The unit dosage form can be a packaged preparation, the package containing discrete quantities of preparation, such as packeted tablets, capsules, and powders in vials or ampoules.

The conjugates and compositions of the present invention can be co-administered with other agents. Co-administration includes administering the conjugate or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the conjugates and compositions of the present invention can each be administered once a day, or two, three, or more times per day so as to provide the preferred dosage level per day.

In some embodiments, co-administration can be accomplished by co-formulation, i.e., preparing a single pharmaceutical composition including the conjugates and compositions of the present invention and any other agent. Alternatively, the various components can be formulated separately.

The conjugates and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, or about 50 mg to about 250 mg. Suitable dosages also include about 1 mg, 5, 10, 20, 30, 40, 50, 60, 70, 80, 90, 100, 200, 300, 400, 500, 600, 700, 800, 900 or 1000 mg. The composition can also contain other compatible therapeutic agents. The conjugates described herein can be used in combination with one another, with other active agents known to be useful in modulating a glucocorticoid receptor, or with adjunctive agents that may not be effective alone, but may contribute to the efficacy of the active agent.

In some embodiments, a composition of the present invention is for use in a method of treating uveitis as described herein.

### IV. Methods of Treatment

In some embodiments, the present invention relates to a method and/or use comprising a conjugate or a composition as described herein for the treatment of uveitis in a subject in need thereof. Uveitis is an eye disease that occurs when the middle layer of the eyeball is inflamed, red and/or swollen. This layer, called the uvea, has many blood vessels that nourish the eye. Uveitis can damage vital eye tissue, leading to permanent vision loss. The uveitis can be anterior uveitis, intermediate uveitis, and/or posterior uveitis.

In some embodiments, the method of the present invention is a method for treating uveitis in a subject in need thereof, comprising administering to the subject a therapeutically effective amount of a conjugate of Formula V:

(X—Y)ₙ—Z (V),
wherein
each X is independently an anti-inflammatory peptide having a molecular weight of from about 5 kDa to about 200 kDa;
each Y is an organic linker;
Z is a hyaluronic acid polymer having a molecular weight of from about 0.1 MDa to about 3 MDa; and
subscript n is an integer of from 1 to 1000.

In some embodiments, each X is independently an anti-TNF-α peptide or an anti-interleukin-1β peptide.

In some embodiments, each X is a monoclonal IgG, an IgG fragment, single chain scFv, single-domain heavy-chain VHH, adnectin, affibody, anticalin, DARPin, or an engineered Kunitz-type inhibitor. In some embodiments, each X is a monoclonal IgG. In some embodiments, each X is an IgG fragment. In some embodiments, each X is a single-domain heavy-chain VHH. In some embodiments, each X is a DARPin.

In some embodiments, each X is a peptide having an amino acid sequence comprising any one of SEQ ID NOS: 61-73, 81-85, 91-98, 101-109, 111-118, and 145-154.

In some embodiments, each X is a peptide having an amino acid sequence comprising:

(SEQ ID NO: 101)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSPSTPPTPS PSTPPGGCDD
DDK,
(SEQ ID NO: 102)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSAEAAAKEA AAKEAAAKAG
C,
(SEQ ID NO: 103)
QVQLQDS GGGLVQAGGS LRLSCAASGG TFSSIIMAWF RQAPGKEREF
VGAVSWSGGT TVYADSVLGR FEISRDSARK SVYLQMNSLK PEDTAVYYCA
ARPYQKYNWA SASYNVWGQG TQVTVSSAEA AAKEAAAKEA AAKAGC,
(SEQ ID NO: 104)
QVQLQES GGGLVQAGGS LRLSCAASGG TFSSIIMAWF RQAPGKEREF
VGAVSWSGGT TVYADSVKGR FTISRDSARK SVYLQMNSLK PEDTAVYYCA
ARPYQKYNWA SASYNVWGQG TQVTVSSAEA AAKEAAAKEA AAKAGC,
(SEQ ID NO: 105)
CGGGVDNKFN KEVGWAFGEI GALPNLNALQ FRAFIISLWD DPSQSANLLA
EAKKLNDAQA PK,
or
(SEQ ID NO: 106)
EIVMTQS PSTLSASVGD RVIITCQASQ SIDNWLSWYQ QKPGKAPKLL
IYRASTLASG VPSRFSGSGS GAEFTLTISS LQPDDFATYY CQNTGGGVSI
AFGQGTKLTV LGGGGGSGGG GSGGGGSGGG GSEVQLVESG GGLVQPGGSL
RLSCTASGFS LSSAAMAWVR QAPGKGLEWV GIIYDSASTY YASWAKGRFT
ISRDTSKNTV YLQMNSLRAE DTAVYYCARE RAIFSGDFVL WGQGTLVTVS
SSPSTPPTPS PSTPPGGC.

Each peptide can be linked to the biocompatible polymer by a variety of organic linkers generally known in the art for forming antibody-drug conjugates, such as those provided by Conju-Probe or BroadPharm of San Diego, CA or Creative Biolabs of Shirley, NY. Methods for forming bioconjugate bonds are described in Bioconjugate Techniques, 3^{rd} Edition, Greg T. Hermanson. The organic linkers can be reactive with amines, carbonyls, carboxyl and activated esters, can react via Click-chemistry (with or without copper), or be reactive with thiols.

Representative organic linkers include an amide or disulfide, or are formed from a reactive group such as succinic anhydride, succinimide, N-hydroxy succinimide, N-chlorosuccinimide, N-bromosuccinimide, maleic anhydride, maleimide, hydantoin, phthalimide, and others. The organic linkers useful in the present invention are small and generally have a molecular weight from about 100 Da to about 500 Da containing two functional groups consisting of a maleimide and either an amine or hydrazide. In some embodiments, the peptide is covalently linked to the polymer via a sulfide bond and an organic linker having a molecular weight of from about 100 Da to about 500 Da. In some embodiments, the organic linker has a molecular weight of from about 100 Da to about 300 Da. In some embodiments, the organic linker comprises a succinimide. In some embodiments, the organic linker is formed using N-beta-maleimidopropionic acid hydrazide (BMPH), N-epsilon-maleimidocaproic acid hydrazide (EMCH), N-aminoethylmaleimide, N-kappa-maleimidoundecanoic acid hydrazide (KUMH), hydrazide-PEG2-maleimide, amine-PEG2-maleimide, hydrazide-PEG3-maleimide, or amine-PEG3-maleimide.

In some embodiments, the organic linker has the structure:

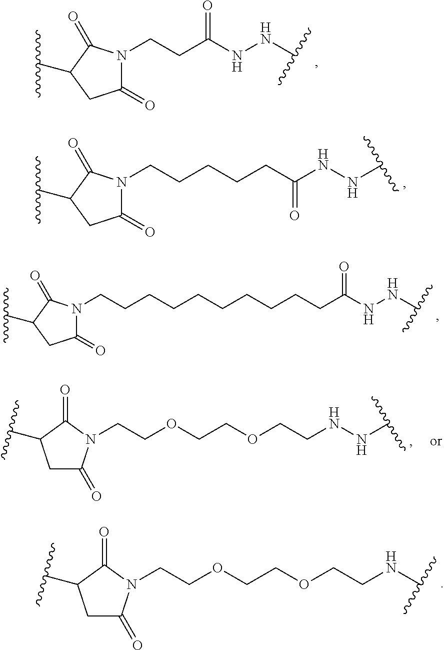

In some embodiments, the organic linker can be N-epsilon-maleimidocaproic acid hydrazide (EMCH):

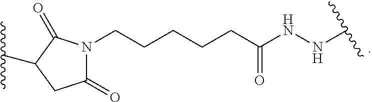 [SMILES: *NNC(=O)CCCCCN1C(=O)CC(*)C1=O]

In some embodiments, the organic linker has the structure:

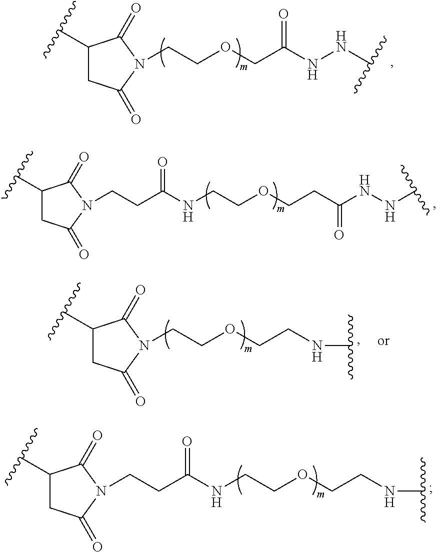

and
subscript m is an integer from 1 to 300. In some embodiments, subscript m is an integer from 1 to 100.

In some embodiments, the organic linker has the structure:

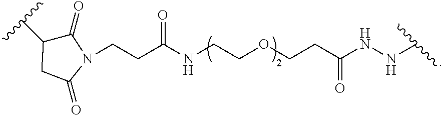

The organic linker with the above structure is known as MP2H.

In some embodiments, each Y is an organic linker having the structure:

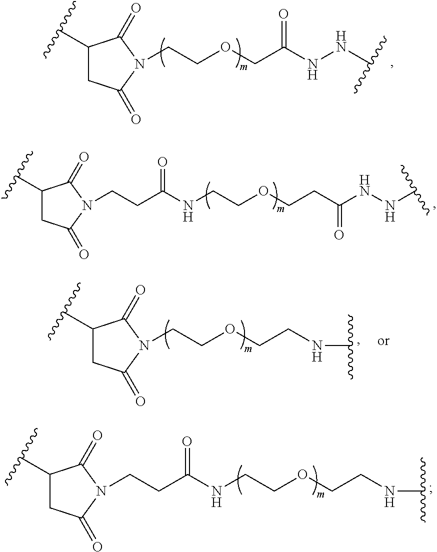

and
subscript m is an integer of from 1 to 300.

In some embodiments, Z has a molecular weight of from about 0.4 MDa to about 2 MDa. In some embodiments, Z has a molecular weight of from about 0.7 MDa to about 1.5 MDa. In some embodiments, Z has a molecular weight of about 0.8 MDa.

In some embodiments, the conjugate of Formula V has the structure of Formula Va:

(X¹—X²—Y)ₙ—Z (Va),
wherein
each X¹ is an an anti-inflammatory peptide having a molecular weight of from about 5 kDa to about 200 kDa;
each X² is a peptide linker that comprises an alpha-helix;
each Y is an organic linker having the structure:

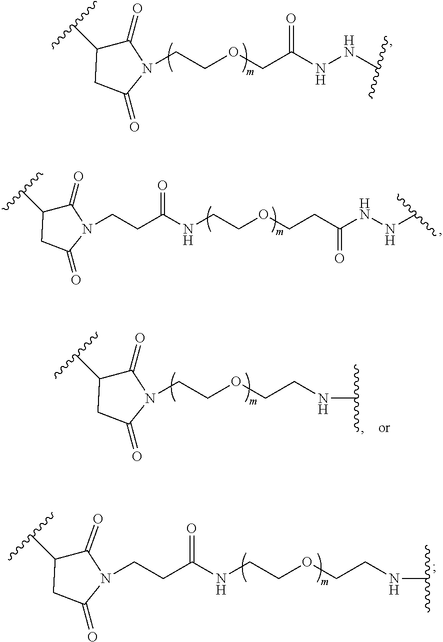

Z is a hyaluronic acid polymer having a molecular weight of from about 0.1 MDa to about 3 MDa; and
subscript m is an integer of from 1 to 300.

In some embodiments, each X¹ is independently an anti-TNF-α peptide or an anti-interleukin-1β peptide.

In some embodiments, each X² is a peptide linker having an amino acid sequence comprising:

(SEQ ID NO: 21)
AEAAAKEAAAKEAAAKAGC,
(SEQ ID NO: 22)
AEEEKRKAEEEKRKAEEEAGC,
(SEQ ID NO: 23)
AEEEKRKAEEEKRKAEEEKRKAEEEAGC,
(SEQ ID NO: 24)
AEEEEKKKKEEEEKKKKAGC,
(SEQ ID NO: 25)
AEAAAKEAAAKAGC,
(SEQ ID NO: 26)
PSRLEEELRRRLTEGC,
or
(SEQ ID NO: 27)
AEEEEKKKQQEEEAERLRRIQEEMEKERKRREEDEERRRKEEEER
RMKLEMEAKRKQEEEERKKREDDEKRKKKAGC.

In some embodiments, each X¹ is a peptide having an amino acid sequence comprising SEQ ID NO: 107, and each X² is a peptide linker having an amino acid sequence comprising SEQ ID NO: 21. In some embodiments, each X¹ is a peptide having an amino acid sequence comprising SEQ ID NO: 108, and each X² is a peptide linker having an amino acid sequence comprising SEQ ID NO: 21. In some embodiments, each X¹ is a peptide having an amino acid sequence comprising SEQ ID NO: 109, and each X² is a peptide linker having an amino acid sequence comprising SEQ ID NO: 21.

In some embodiments, the conjugate of Formula V is a random polymer of Formula VI:

(X—Y—Z¹)ₙ—(Z²)ₚ—(Z³)_{q} (VI),
having a molecular weight of from about 0.1 MDa to about 3 MDa;
wherein
each X is independently an anti-inflammatory peptide having a molecular weight of from about 5 kDa to about 200 kDa;
each Y is an organic linker;
each X—Y—Z¹ moiety has the structure:

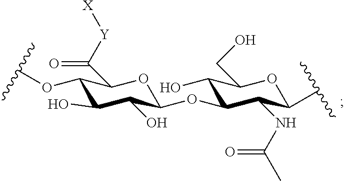

each Z² has the structure:

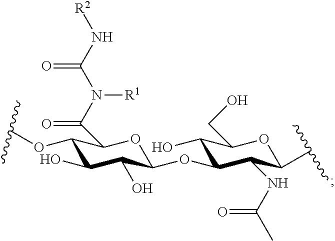

each Z³ independently has the structure:

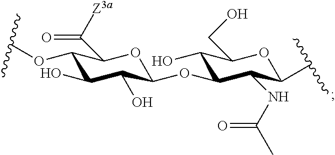

each R¹ and R² is independently C₁-C₆ alkyl, —(C₁-C₆ alkyl)-NR³R⁴, or C₅-C₈ cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

In some embodiments, each R¹ and R² is independently C₁-C₃ alkyl or —(C₁-C₃ alkyl)-NR³R⁴. In some embodiments, each R¹ and R² is ethyl or —(CH₂)₃—NMe₂. In some embodiments, each R¹ is ethyl; and each R² is —(CH₂)₃—NMe₂. In some embodiments, each R¹ is —(CH₂)₃—NMe₂; and each R² is ethyl.

In some embodiments, each R³ and R⁴ is independently C₁-C₃ alkyl.

In some embodiments, preparing the conjugates of the present invention comprises covalently attaching the organic linker to the biocompatible polymer and then covalently attaching the peptide to the organic linker. In some embodiments, after preparing the conjugate of the present invention, unreacted organic linker is present on the biocompatible polymer. The structure of the unreacted organic linker depends on the organic linker and would be understood by a person skilled in the art.

Representative unreacted organic linkers include, but are not limited to,

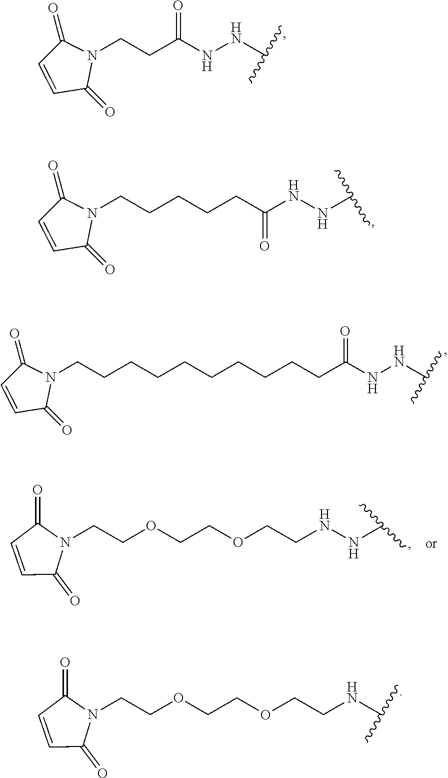 [SMILES: *NNC(=O)CCN1C(=O)C=CC1=O ; *NNC(=O)CCCCCN1C(=O)C=CC1=O ; *NNC(=O)CCCCCCCCCCN1C(=O)C=CC1=O ; *NNCCOCCOCCN1C(=O)C=CC1=O ; *NCCOCCOCCN1C(=O)C=CC1=O]

In some embodiments, the unreacted organic linker has the structure:

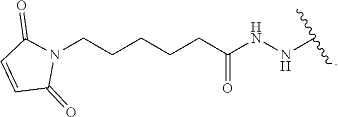 [SMILES: *NNC(=O)CCCCCN1C(=O)C=CC1=O]

In some embodiments, the unreacted organic linker has the structure:

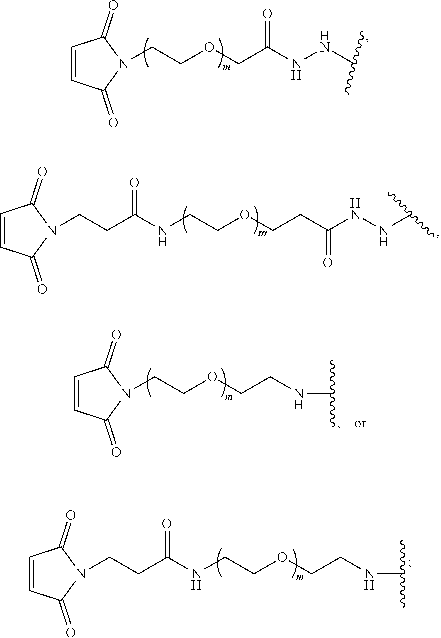

wherein subscript m is an integer of from 1 to 300. In some embodiments, subscript m is an integer from 1 to 100.

In some embodiments, the unreacted organic linker has the structure:

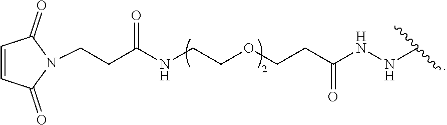

In some embodiments, subscript n is an integer of from 1 to 1500 and less than about 15% of the sum of subscripts n, p, and q; subscript p is an integer of from 1 to 1000 and less than about 10% of the sum of subscripts n, p, and q; and subscript q is an integer of from 100 to 10000. In some embodiments, subscript n is an integer of from 1 to 1000 and less than about 10% of the sum of subscripts n, p, and q; subscript p is an integer of from 1 to 800 and less than about 8% of the sum of subscripts n, p, and q; and subscript q is an integer of from 100 to 10000. In some embodiments, subscript n is an integer of from 10 to 450 and less than about 15% of the sum of subscripts n, p, and q; subscript p is an integer of from 1 to 300 and less than about 10% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 240 and less than about 8% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 60 and less than about 2% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 30 and less than about 1% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 15 and less than about 0.5% of the sum of subscripts n, p, and q; and subscript q is an integer of from 1000 to 3000.

In some embodiments, the uveitis is chronic uveitis. In some embodiments, the uveitis is chronic non-infectious uveitis.

In some embodiments, the method comprises intravitreal administration. In some embodiments, the method comprises multiple administrations of the conjugate. In some embodiments, the method comprises administering the conjugate every month, every two months, or every three months. In some embodiments, the method comprises administering the conjugate twice or three times yearly. In some embodiments, the method comprises administering the conjugate yearly.

In some embodiments, the method of the present invention is a method for treating chronic non-infectious uveitis in a subject in need thereof, comprising administering to the subject a therapeutically effective amount of the conjugate that is a random polymer of Formula VI:

(X—Y—Z¹)ₙ—(Z²)ₚ—(Z³)_{q} (VI),
having a molecular weight of from about 0.1 MDa to about 3 MDa;
wherein
each X is independently an anti-TNF-α or anti-IL-1β peptide comprising:

(SEQ ID NO: 101)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSPSTPPTPS PSTPPGGCDD
DDK,
(SEQ ID NO: 102)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSAEAAAKEA AAKEAAAKAG
C,
(SEQ ID NO: 103)
QVQLQDS GGGLVQAGGS LRLSCAASGG TFSSIIMAWF RQAPGKEREF
VGAVSWSGGT TVYADSVLGR FEISRDSARK SVYLQMNSLK PEDTAVYYCA
ARPYQKYNWA SASYNVWGQG TQVTVSSAEA AAKEAAAKEA AAKAGC,
(SEQ ID NO: 104)
QVQLQES GGGLVQAGGS LRLSCAASGG TFSSIIMAWF RQAPGKEREF
VGAVSWSGGT TVYADSVKGR FTISRDSARK SVYLQMNSLK PEDTAVYYCA
ARPYQKYNWA SASYNVWGQG TQVTVSSAEA AAKEAAAKEA AAKAGC,
(SEQ ID NO: 105)
CGGGVDNKFN KEVGWAFGEI GALPNLNALQ FRAFIISLWD DPSQSANLLA
EAKKLNDAQA PK,
or
(SEQ ID NO: 106)
EIVMTQS PSTLSASVGD RVIITCQASQ SIDNWLSWYQ QKPGKAPKLL
IYRASTLASG VPSRFSGSGS GAEFTLTISS LQPDDFATYY CQNTGGGVSI
AFGQGTKLTV LGGGGGSGGG GSGGGGSGGG GSEVQLVESG GGLVQPGGSL
RLSCTASGFS LSSAAMAWVR QAPGKGLEWV GIIYDSASTY YASWAKGRFT
ISRDTSKNTV YLQMNSLRAE DTAVYYCARE RAIFSGDFVL WGQGTLVTVS
SSPSTPPTPS PSTPPGGC;

each Y is an organic linker having the structure:

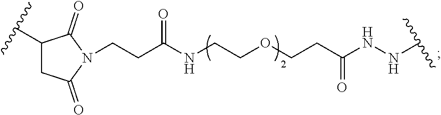

each X—Y—Z¹ moiety has the structure:

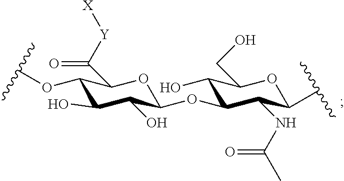

each Z² has the structure:

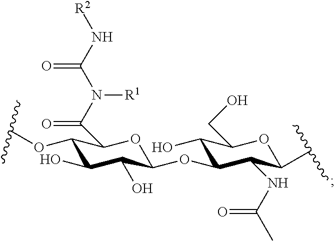

each Z³ independently has the structure:

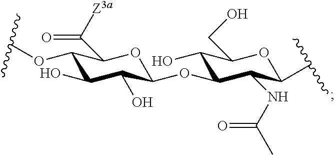

each R¹ and R² is independently C₁-C₆ alkyl, —(C₁-C₆ alkyl)-NR³R⁴, or C₅-C₈cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

In some embodiments, the random polymer of Formula VI has a molecular weight of from about 0.4 MDa to about 2 MDa. In some embodiments, the random polymer of Formula III has a molecular weight of from about 0.7 MDa to about 1.5 MDa. In some embodiments, the random polymer of Formula III has a molecular weight of about 0.8 MDa.

In some embodiments, each X is a peptide having an amino acid sequence comprising SEQ ID NO: 101. In some embodiments, each X is a peptide having an amino acid sequence comprising SEQ ID NO: 102. In some embodiments, each X is a peptide having an amino acid sequence comprising SEQ ID NO: 103. In some embodiments, each X is a peptide having an amino acid sequence comprising SEQ ID NO: 104. In some embodiments, each X is a peptide having an amino acid sequence comprising SEQ ID NO: 105. In some embodiments, each X is a peptide having an amino acid sequence comprising SEQ ID NO: 106.

In some embodiments, a use of the present invention comprises the preparation of a medicament for a method of treating uveitis as described herein.

In some embodiments, the subject is a human.

In some embodiments, a use of the present invention is a use for treating uveitis comprising a conjugate or pharmaceutical composition as described herein.

In some embodiments, a pharmaceutical composition of the present invention is a pharmaceutical composition for use in treating uveitis comprising a conjugate as described herein.

In some embodiments, a conjugate of the present invention is a conjugate for use in treating uveitis as described herein.

### V. EXAMPLES

Certain abbreviations and acronyms are used in describing the experimental details. Although most of these would be understood by one skilled in the art, the Table below contains a list of many of these abbreviations and acronyms.

TABLE 1
List of abbreviations and acronyms.

| Abbreviation | Meaning |
|---|---|
| aH | alpha-helix |
| BLI | biolayer interferometry |
| CBB | Coomassie brilliant blue |
| Da | daltons |
| DLS | dynamic light scattering |
| DMSO | dimethyl sulfoxide |
| DMTMM | 4-(4,6-dimethoxy-1,3,5-triazin-2-yl)-4-methyl |
| | morpholinium chloride |
| DPBS | Dulbecco's phosphate buffered saline |
| DTT | dithiothreitol |
| EAU | experimental autoimmune uveoretinitis |
| EDC | 1-ethyl-3-(3-dimethylaminopropyl)carbodiimide |
| EDTA | ethylenediaminetetraacetic acid |
| EIU | endotoxin-induced uveitis |
| ELISA | enzyme-linked immunosorbent assay |
| FPLC | fast protein liquid chromatography |
| HA or HyA | hyaluronic acid |
| IL | interleukin |
| ITV | intravitreal |
| kDa | kilodaltons |
| MDa | megadaltons |
| MES | 2-(N-morpholino)ethanesulfonic acid |
| MVP | multivalent protein |
| MW | molecular weight |
| MWCO | molecular weight cutoff |
| NHS | N-hydroxysuccinimide |
| PBS | phosphate buffered saline |
| RPM | revolutions per minute |
| RT | room temperature |
| SEC | size-exclusion chromatography |
| SEC MALS | size-exclusion chromatography multi-angle light |
| | scattering |
| TCEP | tris(2-carboxyethyl)phosphine |
| TNFα | tumor necrosis factor alpha |

### Example 1. Preparation of Peptides

Biologically active peptides were prepared optionally with a C-terminal peptide linker for attachment to the polymer.

TABLE 2
Peptides

| SEQ ID NO: | Protein | Type |
|---|---|---|
| 101 | Hu_anti-TNFα-rigid | VHH |
| 102 | Hu_anti-TNFα-aH | VHH |
| 103 | mu_anti-TNFα-aH | VHH |
| 104 | mu_anti-TNFα_3Mut-aH | VHH |
| 105 | anti-TNFα | affibody |
| 106 | anti-IL-1β-rigid | scFv |
| 107 | Hu_anti-TNFα | VHH |
| 108 | Hu_anti-TNFα | VHH |
| 109 | Hu_anti-TNFα | VHH |

### Example 2. Preparation of Purified Thiol Reactive Hyaluronic Acid Conjugate Intermediates

Hyaluronic acid (HA, 830 kDa) was suspended in water or 0.1 M 2-(N-morpholino)ethanesulfonic acid buffer pH 5.7 at 4 mg/mL by gentle rotation or mixing with nutation overnight at RT. To 3 mg (3.6 nmol, amount will vary based on polymer composition and MW) of HA in solution is added hydroxybenzotriazole (HOBt) hydrate as a ˜5-100 mg/mL stock solution in DMSO, thiol reactive linker agent (e.g., hydrazide-X-thiol-reactive-group or amine-X-thiol-reactive-group, for example, MP2H or EMCH) in 10-100% DMSO (10-100 mg/mL stock), and a coupling agent (1-ethyl-3-(3-dimethylaminopropyl)carbodiimide hydrochloride (EDC) or 4-(4,6-Dimethoxy-1,3,5-triazin-2-yl)-4-methylmorpholinium chloride) as a ˜1-0.05 g/mL stock in water or 0.1 M MES buffer pH 5.7. The molar equivalents for each reactant per mole of HA and per carboxylate for different methods of performing the reaction, and example methods are described in the table below:

TABLE 3
Relative Ratios of Coupling Agent,
Catalyst, and Linkers in Methods

| Reactant | Method 1 | Method 2 |
|---|---|---|
| Coupling agent | 500-750 | 9500-12000 |
| (EDC) | | |
| HOBt | 3000 | 50 |
| Linker agent | 3000 | 500-1000 |

Solution was mixed with gentle pipetting between each reagent addition and the final reaction volume was raised to 1 mL with buffer. The final mixture was allowed to react at room temperature for 45 min to 2 h with nutating mixer depending on Method. After the reaction, the thiol reactive biopolymer was purified using 7 kDa MWCO 5-10 mL Zeba desalting spin column equilibrated with 10% v/v glycerol pH 6.5 DPBS, and 0.01% v/v polysorbate 20 (optional), loaded with crude reaction at 20% volume of resin. The desired intermediate was eluted into clean conical tube using centrifuge at RT, elution time ˜25-60 minutes. The intermediate was used immediately for reaction with thiol or aliquotted and flash frozen on dry ice. Maleimide concentration and number of modifications per polymer was determined using UV absorbance, NMR, or a modified Ellman's reaction assay.

Alternatively, reaction pH or equivalents of hydrazide linker, catalyst, and coupling agent (EDC) were altered higher or lower to increase or decrease the number of thiol reactive small molecule linkers covalently linked per biopolymer (valency).

Alternative coupling reagents can be used in place of EDC and HOBt such as DMTMM or oxyma. Activated biopolymer intermediate can also be purified away from reactants using size exclusion chromatography, other desalting columns, tangential flow filtration, ion exchange chromatography, dialysis, or alcohol/acetone precipitation.

### Example 3. Conjugate Preparation

A fixed concentration of peptide was combined with the polymer at various defined feed ratios in PBS and allowed to react at either 4° C. or ambient temperature for at least 4 and 2 hours respectively with rotation or nutating mixing (most reactions are ran at RT to improve solubility). Before the conjugation reaction, 10-100 equivalents of a reducing agent such as DTT or TCEP HCl were added per protein equivalent to reduce any disulfide bridging between peptides. This was removed from the protein solution prior to conjugation by a desalting column or buffer exchange or was added to the conjugation reaction directly in the form of TCEP immobilized on polymeric beads. During the conjugation reaction, one or more of the following was added to improve the reaction efficiency: 0.5-10 mM EDTA to minimize free thiol oxidation, tween20, carbohydrate, or glycerol to stabilize protein and/or help reduce non-specific interactions between protein and activated biopolymer, increased or decreased salt concentration to stabilize protein and/or help reduce non-specific interactions between protein and activated biopolymer. Unreacted peptide was removed from the peptide-polymer conjugates by one or more of the following methods: dialysis with 50-1000 kDa MWCO against an appropriate buffer (pH should be >1 unit above or below the pI of peptide) for two times for 4 hours each and once for at least 4 hours at 4° C.-room temperature, tangential flow filtration against DPBS pH 6-8, or 50 mM tris 150 mM NaCl pH 8-8.5 with EDTA and tween or other additives like trehalose, depending on peptide, FPLC polishing using a size exclusion column, FPLC polishing with an affinity chromatography column designed to bind the polymer component of the conjugate, or selective precipitation of the conjugates. If reaction efficiency was high enough (<4% unreacted protein present) purification may not be necessary.

Alternatively, to each solution of activated polymer, the peptide was added at a suitable peptide:polymer molar feed ratio and Tween-20 to a final concentration of 0.01%-0.03% (optional). The solution was allowed to react for 2 hours to overnight while agitating by rotation (˜5 RPM) or nutation at ambient temperatures. Unreacted peptides were removed by dialysis using 100-1000 kDa MWCO membranes against phosphate buffered saline or equivalent citrate or succinate buffered saline (pH and buffer salt used depends on peptide) with 0.01-0.03% Tween-20 (optional) for three to five times for 4-18 hours each at 4° C.-room temperature. Alternative methods include tangential flow filtration against appropriate buffer or FPLC polishing using a size exclusion column. Additives like tween20, EDTA, and carbohydrates were optionally added to enhance protein stability, depending on peptide.

TABLE 4
Reaction conditions for hyaluronic acid-protein conjugates

| | | | | | | % |
|---|---|---|---|---|---|---|
| | | Reactive | | | | unconjugated |
| Conjugate | | Thiol | | Polymer | Peptide | peptide after |
| ID | Method | (μM) | Peptide | (μM) | (μM) | reaction |
| 1 | 2 | — | anti-TNFα VHH | | | — |
| | | | (human) - rigid | | | |
| | | | (SEQ ID NO: 101) | | | |
| 2 | 2 | 75.85 | anti-TNFα VHH | 0.54 | 38.02 | — |
| | | | (human) - rigid | | | |
| | | | (SEQ ID NO: 101) | | | |
| 3 | 1 | 299.21 | anti-TNFα VHH | 1.85 | 329.20 | — |
| | | | (human) - aH | | | |
| | | | (SEQ ID NO: 102) | | | |
| 4 | 1 | 166.59 | anti-TNFα VHH | 1.23 | 333.17 | 1.8 |
| | | | (human) - aH | | | |
| | | | (SEQ ID NO: 102) | | | |
| 5 | 1 | 190.59 | anti-TNFα VHH | 1.15 | 209.60 | <LOD |
| | | | (mouse) - aH | | | |
| | | | (SEQ ID NO: 103) | | | |
| 6 | 1 | 412.78 | anti-TNFα 3MUT | 3.03 | 454.06 | |
| | | | VHH (mouse) - aH | | | |
| | | | (SEQ ID NO: 104) | | | |
| 7 | 1 | 303.91 | anti-TNFα 3MUT | 2.22 | 334.32 | 13.3 |
| | | | VHH (mouse) - aH | | | |
| | | | (SEQ ID NO: 104) | | | |
| 8 | 2 | — | anti-TNFα | 1.11 | 64.35 | — |
| | | | Affibody | | | |
| | | | (SEQ ID NO: 105) | | | |
| 9 | 2 | — | anti-IL-1β scFv - | 0.09 | 5.36 | — |
| | | | rigid | | | |
| | | | (SEQ ID NO: 106) | | | |
| 10 | 1 | 515.02 | anti-TNFα 3MUT | 3.05 | 566.53 | 12.2 |
| | | | VHH (mouse) - aH | | | |
| | | | (SEQ ID NO: 104) | | | |
| 11 | 1 | 646.13 | anti-TNFα VHH | 3.20 | 710.74 | 3.8 |
| | | | (human) - aH | | | |
| | | | (SEQ ID NO: 102) | | | |
| 12 | 2 | 610.67 | Hu aTNFα 3MUT | 3.03 | 671.73 | 4.7 |
| | | | aH_CYS | | | |
| | | | (SEQ ID NO: 149) | | | |
| 13 | 2 | 610.52 | Hu aTNFα 5MUT | 3.03 | 671.57 | 3.2 |
| | | | aH_CYS | | | |
| | | | (SEQ ID NO: 150) | | | |
| 14 | 2 | 430.36 | Hu aTNFα 7MUT | 2.13 | 473.40 | 3.1 |
| | | | aH_CYS | | | |
| | | | (SEQ ID NO: 151) | | | |

<LOD = below limit of detection

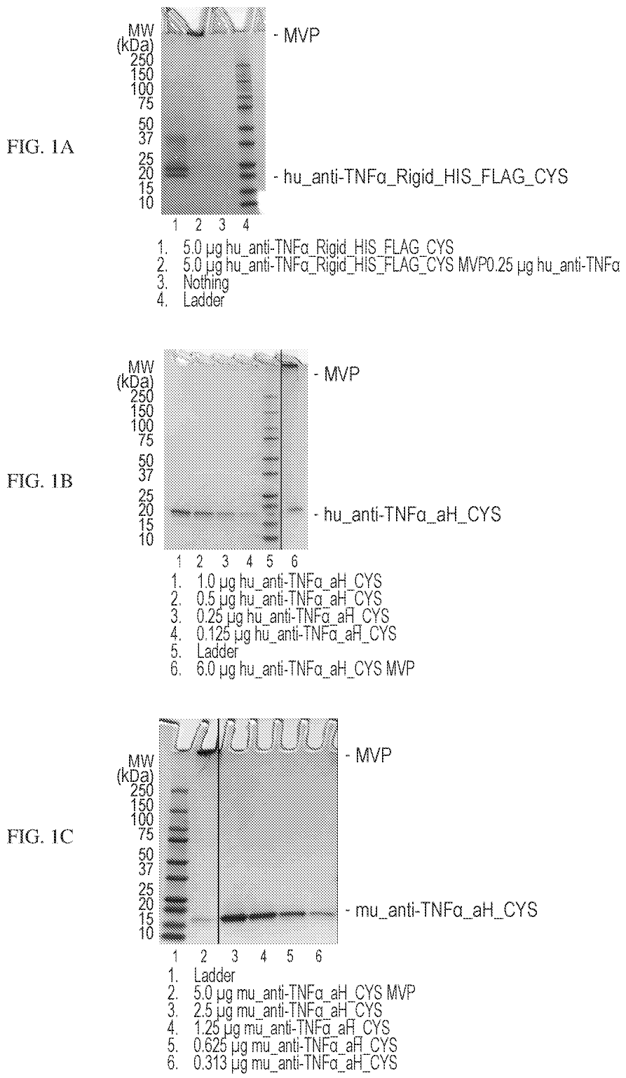

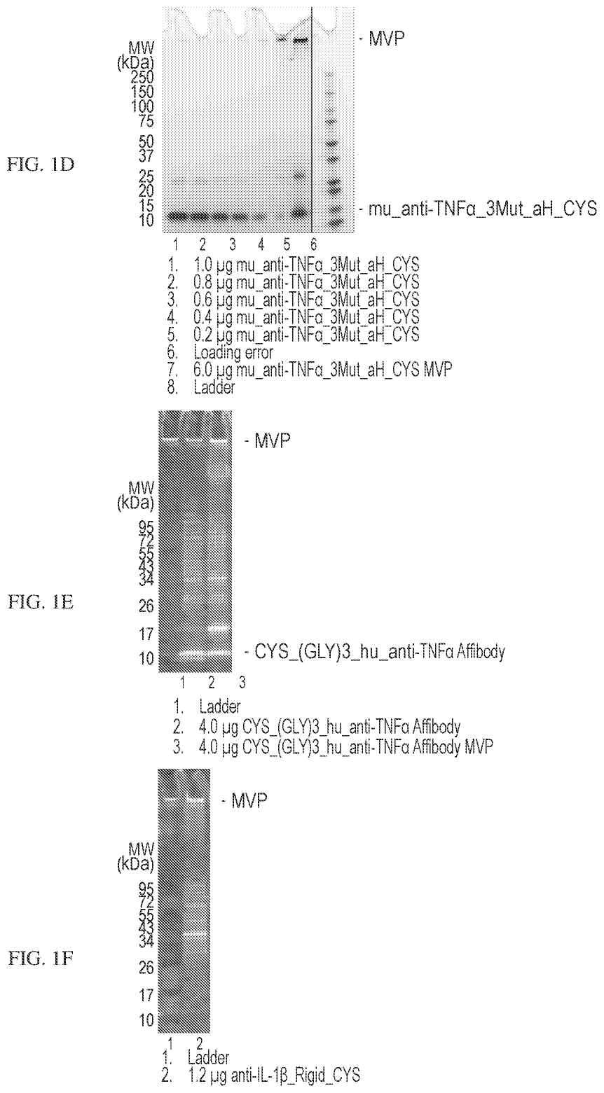

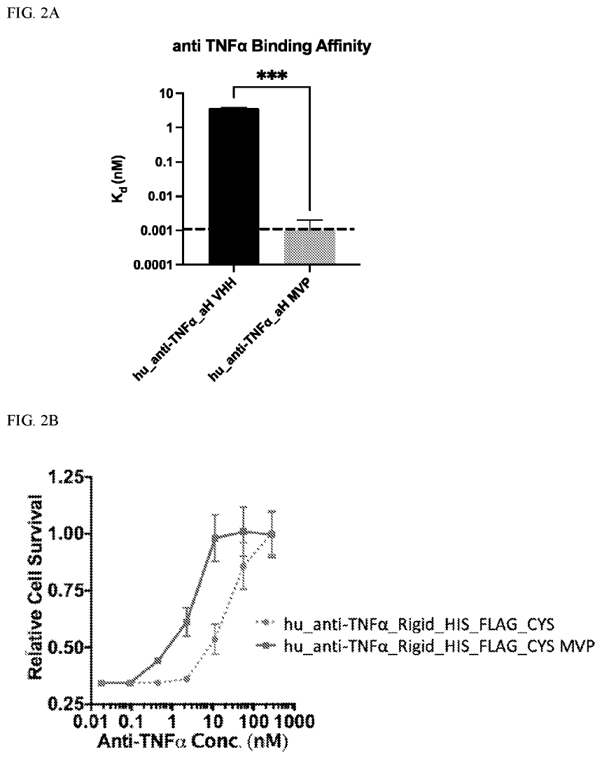

The conjugates in the following table were generated using hyaluronic acid (830 kDa or 850 kDa lots). After purification, the products of the conjugation reactions were analyzed by SDS-PAGE separation to confirm that <20% of the peptide monomer had entered the resolving gel and that >90% of the peptide was present as a macromolecular conjugate at the top of the stacking gel (FIGS. 1A-1F). The reaction products were further analyzed for protein concentration, percent unconjugated peptide, conjugated peptide, valency (molar ratio of conjugated peptide to polymer), and hydrodynamic radius (Rh). Protein concentration was determined based on spectrophotometry at A280, percent unconjugated protein was determined by densitometric analysis of the SDS-PAGE gels, and hydrodynamic radius was measured using dynamic light scattering (DLS).

TABLE 5
Conjugates

| | SEQ | | | Protein | Protein | K_{d} | Rₕ | |
|---|---|---|---|---|---|---|---|---|
| conjugate | ID | Method | Organic | Valency | conc. | (nM, | (nm, | Rₕ |
| # | NO: | # | linker | (UV) | (mg/mL) | BLI) | DLS) | (% PD) |
| 1 | 101 | 2 | EMCH | 9 | 0.094 | — | — | — |
| 2 | 101 | 2 | MP2H | 55 | 0.39 ± | ≤0.001 | | — |
| | | | | | 0.02 | | | |
| 3 | 102 | 1 | MP2H | 65 | 1.24 ± | ≤0.001 | 87.09 ± | 54 ± |
| | | | | | 0.01 | | 9.87 | 8 |
| 4 | 102 | 1 | MP2H | 120 | 2.46 ± | — | — | — |
| | | | | | 0.02 | | | |
| 5 | 103 | 1 | MP2H | 121 | 2.25 ± | ≤0.001 | — | — |
| | | | | | 0.08 | | | |
| 6 | 104 | 1 | MP2H | 51 | 4.77 ± | ≤0.001 | 76.8 ± | — |
| | | | | | 0.02 | | 1.58 | |
| 7 | 104 | 1 | MP2H | 98 | 2.67 ± | ≤0.001 | 76.42 ± | — |
| | | | | | 0.02 | | 5.89 | |
| 8 | 105 | 2 | EMCH | 21 | 0.15 ± | 0.152 | — | — |
| | | | | | 0.01 | | | |
| 9 | 106 | 2 | EMCH | 15 | 0.030 ± | 0.353 | — | — |
| | | | | | 0.00 | | | |
| 10 | 104 | 1 | MP2H | 96.5 | 3.80 ± | ≤0.001 | 99.14 ± | 71.04 ± |
| | | | | | 0.07 | | 5.04 | 13.13 |
| 11 | 102 | 1 | MP2H | 131 | 6.80 ± | ≤0.001 | 135.80 ± | 78.26 ± |
| | | | | | 0.17 | | 10.77 | 21.97 |
| 12 | 149 | 2 | MP2H | 99 | 4.33 ± | ≤0.001 | 70.12 ± | 48.74 ± |
| | | | | | 0.01 | | 4.98 | 14.98 |
| 13 | 150 | 2 | MP2H | 102 | 6.23 ± | ≤0.001 | 71.64 ± | 41.65 ± |
| | | | | | 0.23 | | 7.64 | 14.67 |
| 14 | 151 | 2 | MP2H | 100 | 3.02 ± | 0.0115 | 96.10 ± | 65.02 ± |
| | | | | | 0.31 | | 9.45 | 18.70 |

### Example 4. Potency of MVPs Comprising Anti-Inflammatory Proteins

As one example, we engineered anti-inflammatory proteins containing a peptide linker and thiol linker for conjugation. These antibodies were conjugated to HyA to generate multivalent conjugates at a range of valencies and on different polymer backbones and sizes. Biolayer interferometry (BLI) was performed to quantify binding kinetics for purified MVP as an assessment of bioactivity using a GatorPrime (Gator Bio) or similar instrument and either streptavidin coated probes (Cat #160002) for AVI-tagged ligands or anti-Human Fc (Cat #160003) coated probes for Fc-tagged ligands. All analytes and ligands were diluted in BLI Buffer (1×dPBS, 0.1% w/v BSA and 0.1% v/v polysorbate 20, 0.2 μm filtered). The appropriate ligand for each analyte as noted in Table 6 was first resuspended and stored for long term use according to the manufacturer's directions. The unconjugated analytes were diluted to a top concentration in the range of 5 μM to 1 nM. The multivalent conjugates were diluted to a top concentration of 50-1.0 nM based on the entire multivalent conjugate molecular weight ((protein MW×valency)+polymer mw). The concentration range for each ligand-analyte pair is what demonstrates dose dependence binding affinity in pilot range-finding experiments over a wide titration of concentrations from 10 μM-1 nM.

All reagents were equilibrated to room temperature before use for at least 30 minutes. Two probes per sample (one for kinetic assay and one for ligand free control) were equilibrated in 250 μL BLI buffer (PBS pH 7.4, 0.2% Tween and 0.2% BSA filtered at 0.2 μm) for at minimum 10 min in a Gator Bio Max plate. Ligands were diluted to a fixed concentration of 25-100 nM based on performance in pilot reactions in BLI buffer. Analytes were prepared at the top concentration determined in pilot reactions in BLI buffer and serially diluted 1:3 two to five more times using BLI buffer (Table 6). Black flat-bottom non-coated 96 well plates (Greiner Bio One Cat #655209 or similar) were loaded column-wise with 200 L of ligand, analyte dilutions and one column of BLI buffer for each column of ligand and analyte. One well in each column of analyte was BLI buffer to be used as a blank for reference subtraction. The sample plate was placed in the Gator on a tilted platform set to 25° C. Gator K assay loading and kinetic steps were set up using double reference and step times shown in Table 7. Ligand was loaded until signal reaches between 0.4 and 0.6 nm then returned to buffer column for a baseline measurement for 60-90 s. Next, the kinetic reads were started using the step parameters. When kinetic reads were complete with ligand-loaded probes, a ligand free control was run using new probes that were not loaded with the ligand. The same kinetic assay timing and same sample wells were used that were analyzed with ligand loaded probes. This data was used to correct for any non-specific interactions between the sample and probe.

When kinetic assay was complete, data was analyzed using the Gator software. The raw data was corrected to include the association time after 1 second to 180 seconds. The Y-axis was aligned to the beginning of the association step and interstep correction was used. Savitzky-Goaly filtering of data was used. The samples were set for a double reference by denoting which probes and wells were buffer references in the software. Then, the reference subtraction formula for each assay was edited so that for each assay it was a double reference with the equation of (Kinetic Assay well−Ligand Free Assay well)−(Kinetic Assay buffer reference well−Ligand free assay buffer well). All titrations of the same MVP were grouped by color and the parameters adjusted to a 1:1 binding model that included both association and dissociation with global, Rmax unlinked fitting. The window of interest was moved to include only 100 seconds of dissociation. The binding curve was fitted and checked that the residuals did not vary from the actual curve more than 10%, that the full R² is >0.98 and the Full X² is <3.0. The kinetics and variables K_{D}, Kₒₙ and response were noted.

TABLE 6
BLI Ligands and analyte pairings

| | | | | Catalog |
|---|---|---|---|---|
| BLI Ligand | Tags | Analyte | Supplier | Number |
| Human TNFα | Avi, | anti-TNFα | Acro Biosystems | TNA-H82E3 |
| | His | | | |
| Human IL-1β | Fc | Anti-IL-1β | Acro Biosystems | ILA-H525c |

TABLE 7
BLI method parameters and results specifications
for kinetic quantitation

| | Wells Used | Step time (s) or info |
|---|---|---|
| Parameter | | |
| Probe equilibration | Buffer in Max Plate | >600 |
| Basic Parameters | | 5 Hz, 30 s equilibration, |
| | | 1000 rpm shaking |
| Buffer | | PBS pH 7, 0.2% Tween |
| | | and 0.2% BSA filtered |
| | | at 0.2 μm |
| BLI Experiment | | |
| Parameters | | |
| Baseline | Buffer Column 1 | 60 |
| Ligand loading | 100-25 nM Ligand | When loading |
| | | signal is 0.4-0.6 |
| Baseline | Buffer Column 1 | 90 |
| Association | MVP Sample(s) | 180 |
| Dissociation | Buffer Column 2 | 300 |
| Ligand free control | | |
| (with blank probes) | | |
| Baseline | Buffer Column 2 | 90 |
| Association | MVP Sample(s) | 180 |
| Dissociation | Buffer Column 2 | 300 |

### Example 5. Hydrodynamic Radius of MVPs

Anti-inflammatory agents containing a peptide linker and thiol linker for conjugation were engineered. These agents were conjugated to HyA to generate multivalent conjugates at a range of valencies and on different polymer backbones and sizes. Dynamic light scattering (DLS) was performed to quantify the hydrodynamic radius (Rh) for purified unconjugated protein or MVP as an assessment of size using either a Wyatt Dynapro single cuvette Nanostar, plate reader or similar instrument.

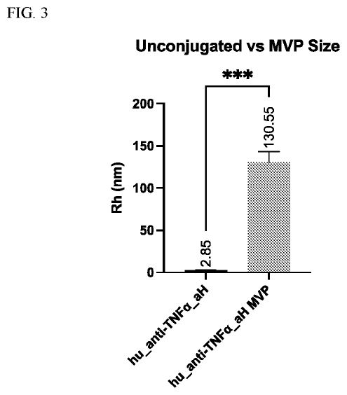

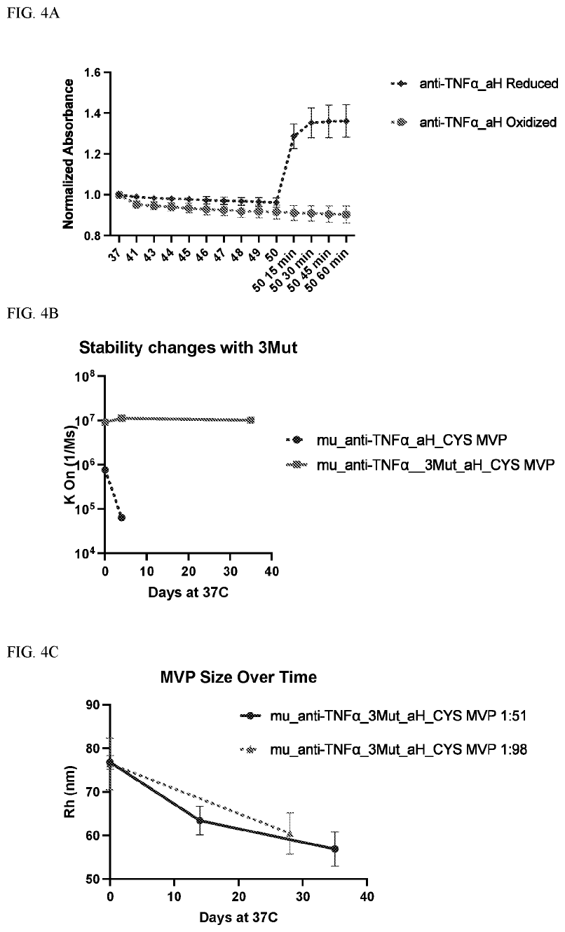

Samples were equilibrated to room temperature for at least 30 minutes. The solution was diluted in 0.1 μm filtered formulation buffer without polysorbate 20 to a final concentration of 100 nM in 100 μL (typically a 1:10 dilution) and mixed by gentle trituration in a 1.5 mL centrifuge tube or up to 30 minutes on a neutator. Large aggregates and dust particles could be removed by spinning the tubes at 5000 g for 5 minutes in a centrifuge. For single cuvette measurements in a NanoStar, a 40 μL sample of the sample solution was loaded into a Wyatt Technology disposable microcuvette (Wyatt Cat #WNDMC) with cap, tapped to remove bubbles, and placed into the instrument for analysis. For multiple readings using the plate reader, 25-35 μL of sample was added to a clear bottomed black well 384 well plate (Corning Cat #P8802-384 or similar). Bubbles in the sample wells were removed. Instrument settings for this and the other sample analyses by DLS in this document are presented in Table 8. DLS acquisition parameters are shown in Table 8 and results specifications are in Table 9. Representative DLS intensity plot for purified, filtered MVP is shown in FIG. 3.

TABLE 8
DLS Acquisition Parameters

| Instrument | | |
|---|---|---|
| Laser wavelength | 664 | nm |
| Laser power | 100% | |
| Auto attenuation | On | |
| Temp | 25° | C. |
| Acquisition time (s) | 5-10 | |
| Acquisition # | 5 | |
| Fixed Parameters | | |
| Correlation function low cutoff | 1.5 | μs |
| Correlation function high cutoff (μs) | 103000 | μs |
| Peak radius low cutoff | 0 | nm |
| Peak radius high cutoff | 1E6 | nm |

| | |
|---|---|
| Analysis type | Dynals |
| Measurement time limit factor | 5 |
| Auto-attenuation time limit | 60 |
| Sample | |
| Mw-R model | Globular proteins |
| Solvent | PBS |
| dn/dc | 0.185 |
| Rg model | Sphere |

TABLE 9
DLS results specification for Multivalent Protein Conjugates

| | Unconjugated | Multivalent |
|---|---|---|
| Peak | Proteins | Protein Conjugates |
| 0-25 nm | >80% intensity | <13% intensity |
| (unconjugated) | | |

| | | | |
|---|---|---|---|
| 25-1000 | nm | >13% intensity | >80% intensity |
| >1000 | nm | <2% intensity | <2% intensity |

### Example 6. Stability of MVPs in a Vitreous Mimetic Buffer

Thermal stability was used as a surrogate to evaluate anti-inflammatory MVPs that may serve as a long term therapeutic and to compare relative stabilities of different constructs. Unconjugated antibodies were diluted to 1.0 mg/mL and anti-inflammatory MVPs to 0.5 mg peptide/mL in formulation buffer. 3×30 μL of each sample was placed in a UV-VIS compatible 384 well plate (Greiner Bio-One Cat #781801 or similar), bubbles were removed, and the plate sealed with UV transparent sealing tape (Greiner Bio-One Cat #676070 or similar). A plate reader with temperature control (Biotek Synergy HTX plate reader with UV/VIS capabilities or similar) was used, and the temperature was increased from 25° C. to 37° C. The plate was incubated for 15 minutes, and the absorbance at 280 nm measured in each well at each step. This program continued until the instrument reached 50° C., where the samples were held for 60 minutes total, measuring the absorbance at 280 nm every 15 minutes. The formulation buffer reference absorbance at 280 nm (A280) value was subtracted from the sample measurement A280 value at each temperature and then they were normalized to the measurement at 37° C. and plotted. These thermal stability plots were compared across different antibodies, mutants and peptide linkers to determine the anti-inflammatory constructs that would be the most resistant to thermal changes and therefore also more likely to be stable enough for a long-term intraocular therapy.

We then used the top performers from the thermal stability experiments in a long-term 37° C. stability studies. The MVPs were synthesized under sterile conditions and diluted to around 0.4 mg/mL in a sterile filtered human vitreous mimetic buffer (see Table 10) or remained in formulation buffer. The samples were either filtered using sterile 0.2 or 5 μm spin filters before use or mixed with 0.010% sodium azide as an anti-microbial agent. Then, several 100-150 μL aliquots of each sample were added to wells of a sterile 96 well plate with one day 0 aliquot reserved at 4° C. The remaining wells were filled with a sterile filtered human vitreous buffer+0.01% sodium azide to minimize evaporation. The plate was incubated in a standard tissue culture incubator at 37° C. with 5% CO₂. At discrete timepoints, one aliquot from each sample was removed from the plate under sterile conditions and analyzed. First, the UV-VIS spectrum of the sample was taken from 200-600 nm in 10 nm steps to monitor any dramatic changes in sample composition. Then, the protein concentration was measured to adjust for any differences in volume that may have occurred. The binding affinity to the appropriate ligand was measured using BLT methods described above using 5-10 nM of MVP as the top concentration. The change in Kₒₙ (association constant) over time was used to assess relative stability over time. To monitor changes in radius over time, the samples were spun for 5 minutes at 5000 g to remove any large aggregates or dust particles and the Rh was measured using DLS methods described above except that the instrument is at 37° C. and without any sample dilution.

TABLE 10
Vitreous Mimetic Buffer Composition

| Component | mg/mL |
|---|---|
| NaCl | 7.14 |
| KCl | 0.38 |
| CaCl₂ 2H₂O | 0.154 |
| MgCl₂ 6H₂O | 0.2 |
| dibasic NaPhosphate (NaH₂PO₄) | 0.42 |
| NaHCO₃ | 2.1 |
| Dextrose | 0.92 |
| lactic acid | 0.358 |
| CuSO₄ | 8.28 × 10⁻⁵ |
| ZnSO₄ heptahydrate | 0.000561 |
| FeCl₂ tetrahydrate | 0.000618 |
| Transferrin (2 Fe binding sites) | 0.0878 |
| Reduced Glutathione (GSH) | 0.0154 |

### Example 7. Peptide-Polymer Conjugates Showed Intravitreal Retention

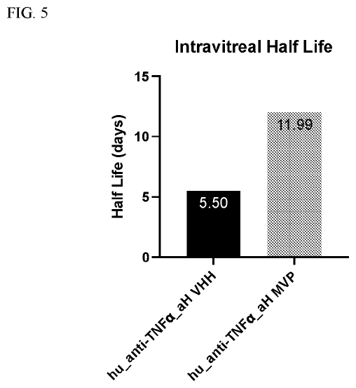

An extended intravitreal retention time of the conjugates was shown in a well-established pharmacokinetics model. New Zealand White rabbits (n=9) were divided into 3 groups randomized by weight. All animals received a 50-μL ITV injections of hu_anti-TNFα_aH MVP in the left eye and the unconjugated VHH in the right eye using a 31 G insulin syringe. Both eyes received an equivalent molar dose of antibody. At 1 hour, 5 days and 10 or days post injection, one group of three rabbits are sacrificed, and their eyes enucleated for analysis of intravitreal VHH. Both eyes were flash frozen, and the vitreous, retina, and aqueous humor were isolated from the frozen eye. Each tissue sample was then homogenized with a bead beater. After homogenization, the VHH concentrations were quantified either using ELISA or by digesting the peptide using trypsin and subjecting the samples to LC/mass spectrometry, or a similar method. Representative results for the extended intravitreal half-life in rabbit eyes after bioconjugation are shown in FIG. 5.

### Example 8. MVP Efficacy in a Rat Model of Uveitis

The efficacy of mu_anti-TNFα_aH_CYS MVPs were validated to provide a treatment effect that can sufficiently reduce the symptoms of uveitis. A rat model of experimental autoimmune uveoretinitis (EAU) was used as a model of chronic posterior uveitis in humans. This model was induced by systemic immunization with the uveitogenic interphotoreceptor retinoid-binding protein (IRBP), and symptoms of uveitis appeared after 9-11 days. Rats were treated intravitreally with 12.5 μg of mu_anti-TNFα_aH_CYS MVP (Conjugate #7). As a positive control, dexamethasone was used.

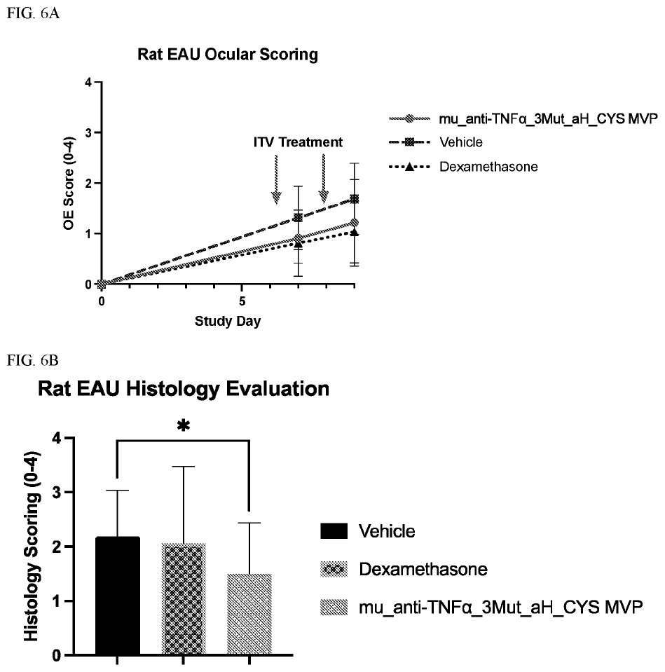

Male Lewis rats were divided into 4 groups (n=8) and randomized by weight. The groups received either mu_anti-TNFα_aH_CYS MVP (Conjugate #7) (using anti-mouse TNFα VHH) at 12.5 g, dexamethasone (40 μg), or vehicle control. On Day 1, rats were immunized by a subcutaneous injection at the base of the tail and in each thigh with 30 μg of bovine IRBP peptide R16 in 0.2 mL of Freund's adjuvant. On days 8 and 10, rat eyes were treated bilaterally with a 5 μL ITV injection of either dose of their assigned treatment. Prior to the start of the study and on days 7, 9, 11, 14, we assessed ocular inflammation by slit lamp microscopy and assigned a clinical EAU score of 0-4 based on the appearance of inflammation. On day 14, animals were euthanized, one eye from each animal was processed for histopathology, and assigned a score of 0-4 based on the appearance of inflammation and cell infiltration. EAU and histopathology were scored based on published standardized scoring systems. The study results are summarized in FIG. 6.

### Example 9. MVP Efficacy in a Second Rat Model of Uveitis

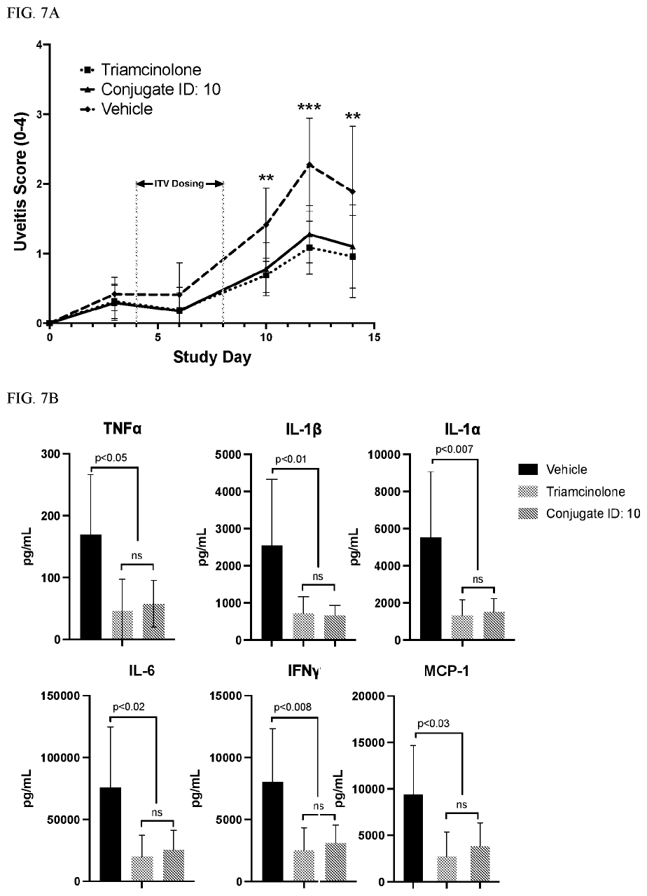

The efficacy of conjugate #10 was validated to provide a treatment effect that can sufficiently reduce the symptoms of uveitis. Male Lewis rats were divided into 4 groups (n=12 for EAU induced groups and n=8 for uninduced control) and randomized by weight. The groups received either conjugate #10 at 19 μg, triamcinolone (40 μg), or vehicle control. On Day 1, rats in the induced groups were immunized by a subcutaneous injection in each flank with 25 μg of interphotoreceptor retinoid-binding protein (IRBP) peptide R16 in 0.1 mL of complete Freund's adjuvant for 50 μg total. On days 4 and 8, rat eyes were treated bilaterally with a 5 μL intravitreal (ITV) injection of conjugate #10 or vehicle control, or 1 μL ITV injection of triamcinolone based on their assigned treatment. Prior to the start of the study and on days 3, 6, 10, 12 and 14, ocular inflammation was accessed by slit lamp microscopy and assigned a clinical EAU score of 0-4 based on the appearance of inflammation. FIGS. 7A-7B show the effect of conjugate #10 was comparable to triamcinolone in reducing ocular inflammation by slit lamp and as measured by inflammatory cytokine or inflammatory regulator levels.

On day 14, animals were euthanized and one eye from each animal was dissected into vitreous and aqueous humor to perform for cytokine analysis. After dissection the aqueous humor from each group was pooled and all tissues were flash frozen. A milliplex Rat Cytokine/Chemokine magnetic bead panel (Millipore Cat #RECYMAG65K27PMX) was used to assess the relative cytokine concentrations in the tissues according to the manufacturer's protocol. Briefly, the ocular tissues were thawed on ice and the sample volume was measured. Then, assay buffer was added to the samples to achieve a final volume of 55 μL for duplicate readings and mixed well. Next, 25 μL of samples, standards or controls was added to the assay plate, mixed with 25 μL of beads and incubated at room temperature for 2 hours. The wells were washed on a magnetic plate washer and incubated with 25 μL of detection antibodies for one hour and then 25 μL of Streptavadin-Phycoerythrin for 30 minutes. Wells were washed on a magnetic plate washer and 125 μL of Sheath Fluid Plus was added per well and then read on the Luminex. The amount of cytokine recovered from each sample was normalized to the volume of tissue recovered and plotted. A graphical analysis of key pro-inflammatory cytokines and inflammatory regulators is shown in FIG. 7B. Select cytokine concentrations are provided in Table 11. No statistical significance was observed in cytokine levels between triamcinolone and conjugate #10 treated rats.

TABLE 11
Cytokine concentration in the vitreous of rat eyes 14 days after uveitis induction

| | Concentration (ng/mL) ± SD | | | Statistical Significance^{§} |
|---|---|---|---|---|
| | Vehicle | | Conjugate | Vehicle Control vs. |
| Cytokine | Control | Triamcinolone | #10 | Conjugate #10 |
| TNFα | 0.169 ± 0.097 | 0.046 ± 0.051 | 0.058 ± 0.038 | * |
| IL-1β | 2.558 ± 1.769 | 0.721 ± 0.445 | 0.667 ± 0.271 | ** |
| IL-1α | 5.532 ± 3.527 | 1.321 ± 0.844 | 1.511 ± 0.709 | ** |
| IL-6 | 75.857 ± 48.960 | 19.936 ± 17.156 | 25.565 ± 15.702 | * |
| IFNγ | 8.048 ± 4.287 | 2.540 ± 1.804 | 3.116 ± 1.436 | ** |
| MCP-1 | 9.432 ± 5.231 | 2.705 ± 2.640 | 3.843 ± 2.499 | * |

^{§}Shown as the p-value of Tukey tests post hoc to ANOVA

### Example 10. MVP Efficacy in a Rabbit TNF-alpha-Induced Model of Uveitis

An anti-TNFα MVP was evaluated in the TNF-α-induced uveitis (EIU) model in rabbits, which involved an ITV injection of human TNF-α that elevated other inflammatory cytokines and induced ocular inflammation characteristic of non-infectious uveitis (NIU) in humans.

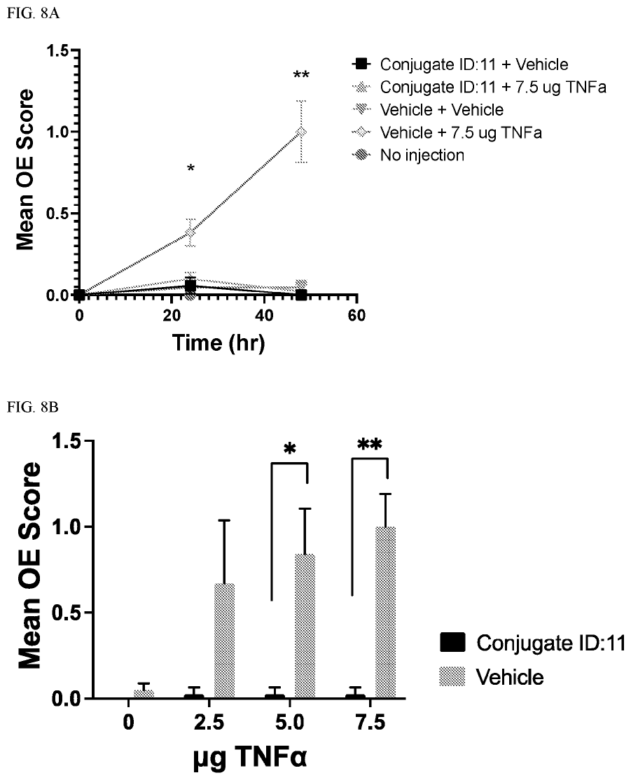

Male New Zealand white (NZW) rabbits were divided into 9 groups randomized by weight (n=3). On day zero, the groups received either the 0.26 mg of hu_anti-TNFα_aH MVP (conjugate #11) (4 groups) or vehicle control (4 groups) administered by bilateral 50-μL ITV injections or no injection (1 group). One day after ITV drug delivery, ocular inflammation was induced by delivering 7.5, 5.0 or 2.5 μg of human TNFα or PBS vehicle control by a unilateral 50-μL ITV injection to the left eye. One uninduced group received no intravitreal injections. Prior to TNFα injection, and at 6, 24 and 48 hours after administering TNFα, inflammation severity was assessed by ocular examination and intraocular pressure was measured using a rebound tonometer. Clinical scores were assigned to each eye based on a published scale. The rabbits were euthanized 48 hours post TNF-α injection. Results are shown in FIGS. 8A-8B.

The left eyes can be dissected into the aqueous and vitreous humor and the vitreous humor can be processed for inflammatory cytokine analysis. Briefly, the dissected vitreous humor is thawed on ice and weighed. The vitreous is gently mixed in a homogenization buffer of PBS 0.05% v.v Tween-20, 1% w/v casein and 0.01% v/v Protease inhibitor cocktail set III (Sigma Catalog number 535140) at a concentration of 500 mg vitreous/mL. Then a solution of bovine testes hyaluronidase (MP Biochemicals catalog #37326-33-3) in PBS with 50 μM MgCl₂ and 100 μM CaCl₂) is added to the vitreous tissue in homogenization buffer at a final concentration of 0.04 mg hyaluronidase/g vitreous tissue. The tissue homogenization reactions are incubated at RT for 1 hour and then 4° C. overnight. Lastly, the homogenized vitreous tissue is spun at 5000 g for 5 minutes to pellet any debris and used for cytokine analysis.

A Milliplex Bovine Cytokine/Chemokine magnetic bead panel (Millipore Cat #BCYT1-33K-12) can be used to assess the relative cytokine concentrations in the tissues according to the manufacturer's protocol. Dissected aqueous humor is thawed and used as is, whereas vitreous humor is homogenized in hyaluronidase as described above. First, 25 μL of samples, standards or controls is added to the assay plate, mixed with 25 μL of beads and incubated at room temperature for 2 hours. The wells are washed on a magnetic plate washer and incubated with 25 μL of detection antibodies for one hour and then 25 μL of Streptavadin-Phycoerythrin for 30 minutes. Wells are washed on a magnetic plate washer and 125 μL of Sheath Fluid Plus is added per well and then read on the Luminex. The amount of cytokine recovered from each sample is normalized to the volume of tissue recovered and plotted.

### Example 11. MVP Efficacy in a Rabbit Endotoxin-Induced Model of Uveitis

hu_anti-TNFα_aH MVP MVPs are evaluated in the endotoxin-induced uveitis (EIU) model in rabbits, which involves an ITV injection of lipopolysaccharide (LPS) that elevates TNFα levels and induces ocular inflammation characteristic of NIU in humans.

NZW rabbits are divided into 4 groups randomized by weight (n=7, 3M/3F, one random). The groups receive either the hu_anti-TNFα aH MVP, a positive control of either adalimumab or triamcinolone, or vehicle control administered by bilateral 50-μL ITV injections. Two groups receive anti-TNFα MVP and one group receives adalimumab at an equivalent molar dose of antigen-binding epitope per eye: 225 μg of total VHH antibody, 1 mg of adalimumab, or 1 mg triamcinolone.

Fifteen days after ITV drug delivery, EIU is induced with 10 g of LPS in 50-μL ITV injections into the left eye of each animal except one of the anti-TNFα MVP groups (durability cohort). 60 days after ITV drug delivery, EIU is induced in the durability cohort using the same method. Prior to LPS injection, and at 6 and 24 hours after administering LPS, inflammation and EIU severity are assessed by ocular examination. EIU clinical scores will be assigned to each eye based on a published scale. The rabbits are euthanized 24 hours post LPS injection. LPS-induced eyes are processed for aqueous humor cell infiltration, inflammatory cytokine analysis, and histopathology to quantify cellular infiltrates. The uninduced right eyes are flash frozen and the anti-TNFα concentrations in the vitreous and aqueous humor are measured.

### Example 12. MVP Durability in a Rabbit Model of TNFα Uveitis

NZW rabbits are divided into 4 groups randomized by weight (n=7, 3M/3F, one random). The groups receive either the hu_anti-TNFα_aH MVP (0.25 mg), triamcinolone (1 mg), or vehicle control administered by bilateral 50-μL ITV injections. Two groups receive anti-TNFα MVP and one group receives triamcinolone.

1-30 days after ITV drug delivery, ocular inflammation is induced by delivering 7.5 μg of human TNFα or PBS vehicle control by a unilateral 50-μL ITV injection to the left eye. One uninduced group received no intravitreal injections. 60 days after ITV drug delivery, EIU is induced in the durability cohort using the same method. Prior to TNFα injection, and at 6, 24 and 48 hours after administering TNFα, inflammation severity is assessed by ocular examination. Intraocular pressure is also measured using a rebound tonometer on a daily basis. Clinical scores are assigned to each eye based on a published scale. The rabbits are euthanized 48 hours post TNFα injection.

The left eyes are dissected into the aqueous and vitreous humor, and the vitreous humor is processed for inflammatory cytokine analysis. Briefly, the dissected vitreous humor is thawed on ice and weighed. The vitreous is gently mixed in a homogenization buffer of PBS 0.05% v/v Tween-20, 1% w/v casein and 0.01% v/v Protease inhibitor cocktail set III (Sigma Catalog number 535140) at a concentration of 500 mg vitreous/mL. Then a solution of bovine testes hyaluronidase (MP Biochemicals catalog #37326-33-3) in PBS with 50 μM MgCl₂ and 100 μM CaCl₂) is added to the vitreous tissue in homogenization buffer at a final concentration of 0.04 mg hyaluronidase/g vitreous tissue. The tissue homogenization reactions are incubated at RT for 1 hour and then 4C overnight. Lastly, the homogenized vitreous tissue is spun at 5000 g for 5 minutes to pellet any debris and used for cytokine analysis.

A Milliplex Bovine Cytokine/Chemokine magnetic bead panel (Millipore Cat4!BCYT1-33K-12) is used to assess the relative cytokine concentrations in the tissues according to the manufacturer's protocol. Dissected aqueous humor is thawed and used and vitreous humor is homogenized in hyaluronidase as described above. First, 25 μL of samples, standards or controls is added to the assay plate, mixed with 25 μL of beads and incubated at room temperature for 2 hours. The wells are washed on a magnetic plate washer and incubated with 25 μL of detection antibodies for one hour and then 25 μL of Streptavadin-Phycoerythrin for 30 minutes. Wells are washed on a magnetic plate washer and 125 μL of Sheath Fluid Plus is added per well and then read on the Luminex. The amount of cytokine recovered from each sample is normalized to the volume of tissue recovered and plotted.

TABLE 12
Sequences

| Name | Sequence |
|---|---|
| blank | SEQ ID NO: 1-4 |
| framework | QVQLVESGGGLVQPGGSLRLSCAASG (SEQ ID NO: 5) |
| region 1 | |
| framework | MGWFRQAPGKEREFVAAI (SEQ ID NO: 6) |
| region 2 | |
| framework | YADSVKGRFTISRDNSKNTVYLQMNSLRPEDTAVYYCAA (SEQ ID NO: |
| region 3 | 7) |
| framework | YWGQGTLVTVSS (SEQ ID NO: 8) |
| region 4 | |
| Nb42 | FAYSTYS (SEQ ID NO: 9) |
| CDR1 | |
| Nb42 | NSGTFRLW (SEQ ID NO: 10) |
| CDR2 | |
| Nb42 | RAWSPYSSTVDAGDFR (SEQ ID NO: 11) |
| CDR3 | |
| blank | SEQ ID NO: 12-14 |
| aTNFa-mu | GTFSSII (SEQ ID NO: 15) |
| CDR1 | |
| aTNFa-mu | SWSGGTTV (SEQ ID NO: 16) |
| CDR2 | |
| aTNFa-mu | RPYQKYNWASASYNV (SEQ ID NO: 17) |
| CDR3 | |
| E1-1 CDR1 | GGSDAGT (SEQ ID NO: 18) |
| E1-1 CDR2 | SWAGTAWR (SEQ ID NO: 19) |
| E1-1 CDR3 | LGSYEMDHH (SEQ ID NO: 20) |
| aH linker | AEAAAKEAAAKEAAAKAGC (SEQ ID NO: 21) |
| AE3K2R(2) | AEEEKRKAEEEKRKAEEEAGC (SEQ ID NO: 22) |
| linker | |
| AE3K2R(3) | AEEEKRKAEEEKRKAEEEKRKAEEEAGC (SEQ ID NO: 23) |
| linker | |
| E4K4(2) | AEEEEKKKKEEEEKKKKAGC (SEQ ID NO: 24) |
| linker | |
| EA3K(2) | AEAAAKEAAAKAGC (SEQ ID NO: 25) |
| linker | |
| Alfa linker | PSRLEEELRRRLTEGC (SEQ ID NO: 26) |
| MyosinVI | AEEEEKKKQQEEEAERLRRIQEEMEKERKRREEDEERRRKEEEERRMK |
| linker | LEMEAKRKQEEEERKKREDDEKRKKKAGC (SEQ ID NO: 27) |
| Spot linker | PDRVRAVSHWSSC (SEQ ID NO: 28) |
| GT9 linker | GTGTGTGTGTGTGTGTGTGC (SEQ ID NO: 29) |
| Modified | TPTTPPTPTPGTPPGGC (SEQ ID NO: 30) |
| Rigid linker | |
| blank | SEQ ID NO: 31-60 |
| Nb42 | QVQLQESGGGSLQAGASLRLSCAASGFAYSTYSMGWFRQVSGKEREG |
| | VATINSGTFRLWYTDSVKGSFTISRDNAKNMLYLQMNSLKPEDTAIYY |
| | CAARAWSPYSSTVDAGDFRYWGQGTQVTVSS (SEQ ID NO: 61) |
| HuNb42 | QVQLVESGGGLVQPGGSLRLSCAASGFAYSTYSMGWFRQAPGKEREA |
| | VATINSGTFRLWYTDSVKGRFTISRDNSKNTLYLQMNSLRAEDTAVYY |
| | CAARAWSPYSSTVDAGDFRYWGQGTLVTVSS (SEQ ID NO: 62) |
| HuNb42 | QVQLVESGGGLVQAGGSLRLSCAASGFAYSTYSMGWFRQAPGKEREA |
| P14A | VATINSGTFRLWYTDSVKGRFTISRDNSKNTLYLQMNSLRAEDTAVYY |
| | CAARAWSPYSSTVDAGDFRYWGQGTLVTVSS (SEQ ID NO: 63) |
| HuNb42 | QVQLVESGGGLVQPGGSLRLSCAASGFAYSTYSMGWFRQAPGKEREA |
| T61A | VATINSGTFRLWYADSVKGRFTISRDNSKNTLYLQMNSLRAEDTAVYY |
| | CAARAWSPYSSTVDAGDFRYWGQGTLVTVSS (SEQ ID NO: 64) |
| HuNb42 | QVQLVESGGGLVQPGGSLRLSCAASGFAYSTYSMGWFRQAPGKEREA |
| S75A | VATINSGTFRLWYTDSVKGRFTISRDNAKNTLYLQMNSLRAEDTAVYY |
| | CAARAWSPYSSTVDAGDFRYWGQGTLVTVSS (SEQ ID NO: 65) |
| HuNb42 | QVQLVESGGGLVQPGGSLRLSCAASGFAYSTYSMGWFRQAPGKEREA |
| L79V | VATINSGTFRLWYTDSVKGRFTISRDNSKNTVYLQMNSLRAEDTAVYY |
| | CAARAWSPYSSTVDAGDFRYWGQGTLVTVSS (SEQ ID NO: 66) |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| | CAARAWSPYSSTVDAGDFRYWGQGTLVTVSS (SEQ ID NO: 67) |
| HuNb42 | QVQLVESGGGLVQPGGSLRLSCAASGFAYSTYSMGWFRQAPGKEREA |
| L121Q | VATINSGTFRLWYTDSVKGRFTISRDNSKNTLYLQMNSLRAEDTAVYY |
| | CAARAWSPYSSTVDAGDFRYWGQGTQVTVSS (SEQ ID NO: 68) |
| HuNb42 | QVQLVESGGGLVQPGGSLRLSCAASGFAYSTYSMGWFRQAPGKEREA |
| T61A A88P | VATINSGTFRLWYADSVKGRFTISRDNSKNTLYLQMNSLRPEDTAVYY |
| | CAARAWSPYSSTVDAGDFRYWGQGTLVTVSS (SEQ ID NO: 69) |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| L115Q | CAARAWSPYSSTVDAGDFRYWGQGTQVTVSS (SEQ ID NO: 70) |
| aTNF-a mu | QVQLQDSGGGLVQAGGSLRLSCAASGGTFSSIIMAWFRQAPGKEREFV |
| | GAVSWSGGTTVYADSVLGRFEISRDSARKSVYLQMNSLKPEDTAVYYC |
| | AARPYQKYNWASASYNVWGQGTQVTVSS (SEQ ID NO: 71) |
| aTNF-a mu | QVQLQESGGGLVQAGGSLRLSCAASGGTFSSIIMAWFRQAPGKEREFV |
| 3MUT | GAVSWSGGTTVYADSVKGRFTISRDSARKSVYLQMNSLKPEDTAVYY |
| | CAARPYQKYNWASASYNVWGQGTQVTVSS (SEQ ID NO: 72) |
| aTNF-a | QVQLQESGGGLVQPGGSLRLSCAASGRTFSDHSGYTYTIGWFRQAPGK |
| VHH | EREFVARIYWSSGNTYYADSVKGRFAISRDIAKNTVDLTMNNLEPEDT |
| | AVYYCAARDGIPTSRSVESYNYWGQGTQVTVSS (SEQ ID NO: 73) |
| blank | SEQ ID NO: 74-80 |
| E1-1 | EVQLQASGGGFVQPGGSLRLSCAASGGGSDAGTMGWFRQAPGKEREF |
| | VSAISWAGTAWRYYADSVKGRFTISRDNSKNTVYLQMNSLRAEDTAT |
| | YYCALGSYEMDHHYWGQGTQVTVSS (SEQ ID NO: 81) |
| E1-1 F11L | EVQLQASGGGLVQPGGSLRLSCAASGGGSDAGTMGWFRQAPGKEREF |
| | VSAISWAGTAWRYYADSVKGRFTISRDNSKNTVYLQMNSLRAEDTAT |
| | YYCALGSYEMDHHYWGQGTQVTVSS (SEQ ID NO: 82) |
| E1-1 S49A | EVQLQASGGGFVQPGGSLRLSCAASGGGSDAGTMGWFRQAPGKEREF |
| | VAAISWAGTAWRYYADSVKGRFTISRDNSKNTVYLQMNSLRAEDTAT |
| | YYCALGSYEMDHHYWGQGTQVTVSS (SEQ ID NO: 83) |
| E1-1 F11L | EVQLQASGGGLVQPGGSLRLSCAASGGGSDAGTMGWFRQAPGKEREF |
| S49A | VAAISWAGTAWRYYADSVKGRFTISRDNSKNTVYLQMNSLRAEDTAT |
| | YYCALGSYEMDHHYWGQGTQVTVSS (SEQ ID NO: 84) |
| E1-1 CDR | EVQLQASGGGFVQPGGSLRLSCAASGRRFSIEAMGWFRQAPGKEREFV |
| | SAIDSGGSTDYADSVKGRFTISRDNSKNTVYLQMNSLRAEDTATYYCA |
| | VIGSSWYGRGLDYWGQGTQVTVSS (SEQ ID NO: 85) |
| blank | SEQ ID NO: 86-90 |
| anti-VEGF | DVQLVESGGGLVQPGGSLRLSCAASGRTFSSYSMGWFRQAPGKEREFV |
| VHH | VAISKGGYKYDAVSLEGRFTISRDNAKNTVYLQINSLRPEDTAVYYCAS |
| | SRAYGSSRLRLADTYEYWGQGTLVTVSS (SEQ ID NO: 91) |
| anti-VEGF | GSDLDKKLLEAARAGQDDEVRILMANGADVNARDSTGWTPLHLAAP |
| DARPIN | WGHPEIVEVLLKNGADVNAADFQGWTPLHLAAAVGHLEIVEVLLKYG |
| | ADVNAQDKFGKTAFDISIDNGNEDLAEILQKAAGGGSGGGS (SEQ ID |
| | NO: 92) |
| anti-VEGF | QVQLVESGGGLVQPGGSLRLSCAASGYAYDTYYMGWFRQAPGKEREG |
| HuNb22 | VAGITSLVSGVAYYKYYTDSVKGRFTISRDNSKNTVDLQMNSLRAEDT |
| 2MUT | AVYYCAASRSGLRARLLRPELYEYWGQGTLVTVSS (SEQ ID NO: 93) |
| anti-VEGF | QVQLVESGGGLVQPGGSLRLSCVASGDTYSSACMGWFRQAPGKEREG |
| HuNb23 | VATICTSTSMRTRYYADSVKGRFTISRDNSKNTVYLQMNSLRAEDTAV |
| 3MUT | YYCATGHTVGSSWRDPGAWRYWGQGTLVTVSS (SEQ ID NO: 94) |
| anti-VEGF | QVQLVESGGGLVQPGGSLRLSCAASGLSYRPGYMGWFRQAPGKEREG |
| HuNb35 | VAIITTGGVTHYADSVKGRFTISRDNSKNTVYLQMNSLRAEDTAVYYC |
| 4MUT | ALANWVQFPLRVDGYKYWGQGTLVTVSS (SEQ ID NO: 95) |
| Hu_aVEGF_ | QVQLVESGGGLVQPGGSLRLSCAASGRTFSSYSMGWFRQAPGKEREFV |
| VHH_3MUT | AAISKGGYKYDAVSLEGRFTISRDNSKNTVYLQMNSLRPEDTAVYYCA |
| | SSRAYGSSRLRLADTYEYWGQGTLVTVSS (SEQ ID NO: 96) |
| Hu_aVEGF_ | QVQLVESGGGLVQPGGSLRLSCAASGRTFSSYSMGWFRQAPGKEREFV |
| VHH_5MUT | AAISKGGYKYDAVSVKGRFTISRDNSKNTVYLQMNSLRPEDTAVYYCA |
| | SSRAYGSSRLRLADTYEYWGQGTLVTVSS (SEQ ID NO: 97) |
| Hu_aVEGF_ | QVQLVESGGGLVQPGGSLRLSCAASGRTFSSYSMGWFRQAPGKEREFV |
| VHH_6MUT | AAISKGGYKYYAVSVKGRFTISRDNSKNTVYLQMNSLRPEDTAVYYCA |
| | SSRAYGSSRLRLADTYEYWGQGTLVTVSS (SEQ ID NO: 98) |
| blank | SEQ ID NO: 99-100 |
| anti-TNFα | QVQLQESGGGLVQPGGSLRLSCAASGRTFSDHSGYTYTIGWFRQAPGK |
| VHH | EREFVARIYWSSGNTYYADSVKGRFAISRDIAKNTVDLTMNNLEPEDT |
| (human)- | AVYYCAARDGIPTSRSVESYNYWGQGTQVTVSSPSTPPTPSPSTPPGGC |
| rigid | DDDDK (SEQ ID NO: 101) |
| anti-TNFα | QVQLQESGGGLVQPGGSLRLSCAASGRTFSDHSGYTYTIGWFRQAPGK |
| VHH | EREFVARIYWSSGNTYYADSVKGRFAISRDIAKNTVDLTMNNLEPEDT |
| (human)- | AVYYCAARDGIPTSRSVESYNYWGQGTQVTVSSAEAAAKEAAAKEAA |
| aH | AKAGC (SEQ ID NO: 102) |
| anti-TNFα | QVQLQDSGGGLVQAGGSLRLSCAASGGTFSSIIMAWFRQAPGKEREFV |
| VHH | GAVSWSGGTTVYADSVLGRFEISRDSARKSVYLQMNSLKFPEDTAVYY |
| (mouse)- | CAARPYQKYNWASASYNVWGQGTQVTVSSAEAAAKEAAAKEAAAK |
| aH | AGC (SEQ ID NO: 103) |
| anti-TNFα | QVQLQESGGGLVQAGGSLRLSCAASGGTFSSIIMAWFRQAPGKEREFV |
| 3MUT | GAVSWSGGTTVY ADSVKGRFTISRDSARKSVYLQMNSLKPEDTAVYY |
| VHH | CAARPYQKYNWASASYNVWGQGTQVTVSSAEAAAKEAAAKEAAAK |
| (mouse)- | AGC (SEQ ID NO: 104) |
| aH | |
| anti-TNFα | CGGGVDNKFNKEVGWAFGEIGALPNLNALQFRAFIISLWDDPSQSANL |
| affibody | LAEAKKLNDAQAPK (SEQ ID NO: 105) |
| anti-IL-1β | EIVMTQSPSTLSASVGDRVIITCQASQSIDNWLSWYQQKPGKAPKLLIYR |
| scFv-rigid | ASTLASGVPSRFSGSGSGAEFTLTISSLQPDDFATYYCQNTGGGVSIAFG |
| | QGTKLTVLGGGGGSGGGGSGGGGSGGGGSEVQLVESGGGLVQPGGSL |
| | RLSCTASGFSLSSAAMAWVRQAPGKGLEWVGIIYDSASTYYASWAKG |
| | RFTISRDTSKNTVYLQMNSLRAEDTAVYYCARERAIFSGDFVLWGQGT |
| | LVTVSSSPSTPPTPSPSTPPGGC (SEQ ID NO: 106) |
| Hu_aTNFa | QVQLVESGGGLVQPGGSLRLSCAASGGTFSSIIMAWFRQAPGKEREFVG |
| Mu_3MUT | AVSWSGGTTVYADSVKGRFTISRDNSKNTVYLQMNSLRPEDTAVYYC |
| | AARPYQKYNWASASYNVWGQGTLVTVSS (SEQ ID NO: 107) |
| Hu_aTNFa | QVQLVESGGGLVQPGGSLRLSCAASGGTFSSIIMAWFRQAPGKEREFVA |
| Mu_5MUT | AISWSGGTTVYADSVKGRFTISRDNSKNTVYLQMNSLRPEDTAVYYCA |
| | ARPYQKYNWASASYNVWGQGTLVTVSS (SEQ ID NO: 108) |
| Hu_aTNFa | QVQLVESGGGLVQPGGSLRLSCAASGRTFSDHSGYTYTMGWFRQAPG |
| Hu_7MUT | KEREFVARIYWSSGNTYYADSVKGRFTISRDNSKNTVYLQMNSLRPED |
| | TAVYYCAARDGIPTSRSVESYNYWGQGTLVTVSS (SEQ ID NO: 109) |
| blank | SEQ ID NO: 110 |
| Hu aEGFR | EVQLVESGGGLVQPGGSLRLSCAASGRTSRSYGMGWFRQAPGKEREFV |
| 3MUT | AGISWRGDSTGYADSVKGRFTISRDNSKNTVDLQMNSLRPEDTAVYYC |
| | AAAAGSAWYGTLYEYDYWGQGTLVTVSS (SEQ ID NO: 111) |
| Hu_aHer2_ | EVQLVESGGGLVQPGGSLRLSCAASGITFMRYAMGWYRQAPGKQREM |
| 3MUT | VASINSGGTTNYADSVKGRFTISRDNSKNTVYLQMNSLRPEDTAVYYC |
| | NARWVKPQFIDNNYWGQGTLVTVSS (SEQ ID NO: 112) |
| Hu_aPD1_ | EVQLVESGGGLVQPGGSLRLSCAASGSIFSIHAMGWFRQAPGKEREFVA |
| 102C3 | AITWSGGITYYEDSVKGRFTISRDNSKNTVYLQMNSLRPEDTAVYYCA |
| 3MUT | ADRAESSWYDYWGQGTLVTVSS (SEQ ID NO: 113) |
| Hu_aPD1_ | EVQLVESGGGLVQPGGSLRLSCAASGSIASIHAMGWFRQAPGKEREFV |
| 102C12 | AVITWSGGITYYADSVKGRFTISRDNSKNTVYLQMNSLRPEDTAVYYC |
| 3MUT | AGDKHQSSWYDYWGQGTLVTVSS (SEQ ID NO: 114) |
| Hu_aPD1_ | EVQLVESGGGLVQPGGSLRLSCAASGSISSIHAMGWFRQAPGKEREFVA |
| 102E2 | AITWSGGITYYADSVKGRFTISRDNSKNTVYLQMNSLRPEDTAVYYCA |
| 3MUT | ADRAQSSWYDYWGQGTLVTVSS (SEQ ID NO: 115) |
| Hu_aPD1_ | EVQLVESGGGLVQPGGSLRLSCAASGSIFSINAMAWFRQAPGKEREFVA |
| 102E8 | LISWSGGSTYYEDSVKGRFTISRDNSKNTVYLQMNSLRPEDTAVYYCA |
| 3MUT | ADRVDSNWYDYWGQGTLVTVSS (SEQ ID NO: 116) |
| Hu_aPD1_ | EVQLVESGGGLVQPGGSLRLSCAASGRAFSSGTMGWFRQAPGKEREFV |
| 102H12 | ASIPWSGGRTYYADSVKGRFTISRDNSKNTVYLQMNSLRPEDTAVYYC |
| 4MUT | AVKERSTGWDFAWGQGTLVTVSS (SEQ ID NO: 117) |
| Hu | EVQLVESGGGLVQPGGSLRLSCAASGRTGTIYSMAWFRQAPGKEREFL |
| aCaffeine | ATIGWSSGITYYMDSVKGRFTISRDNSKNTVYLQMNSLRPEDTAVYYC |
| 3MUT | AATRAYSVGYDYWGQGTLVTVSS (SEQ ID NO: 118) |
| blank | SEQ ID NO: 119-144 |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| aH_CYS | CAARAWSPYSSTVDAGDFRYWGQGTLVTVSSAEAAAKEAAAKEAAA |
| | KAGC (SEQ ID NO: 145) |
| HuNb42 | QVQLVESGGGLVQPGGSLRLSCAASGFAYSTYSMGWFRQAPGKEREA |
| T61A A88P | VATINSGTFRLWYADSVKGRFTISRDNSKNTLYLQMNSLRPEDTAVYY |
| aH_CYS | CAARAWSPYSSTVDAGDFRYWGQGTLVTVSSAEAAAKEAAAKEAAA |
| | KAGC (SEQ ID NO: 146) |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| L115Q | CAARAWSPYSSTVDAGDFRYWGQGTQVTVSSAEAAAKEAAAKEAAA |
| aH_CYS | KAGC (SEQ ID NO: 147) |
| anti-TNFα | CGGGVDNKFNKEVGWAFGEIGALPNLNALQFRAFIISLWDDPSQSANL |
| affibody | LAEAKKLNDAQAPKGGG (SEQ ID NO: 148) |
| Gly | |
| Hu_aTNFa | QVQLVESGGGLVQPGGSLRLSCAASGGTFSSIIMAWFRQAPGKEREFVG |
| Mu_3MUT | AVSWSGGTTVYADSVKGRFTISRDNSKNTVYLQMNSLRPEDTAVYYC |
| aH_CYS | AARPYQKYNWASASYNVWGQGTLVTVSSAEAAAKEAAAKEAAAKA |
| | GC (SEQ ID NO: 149) |
| Hu_aTNFa | QVQLVESGGGLVQPGGSLRLSCAASGGTFSSIIMAWFRQAPGKEREFVA |
| Mu_5MUT | AISWSGGTTVYADSVKGRFTISRDNSKNTVYLQMNSLRPEDTAVYYCA |
| aH_CYS | ARPYQKYNWASASYNVWGQGTLVTVSSAEAAAKEAAAKEAAAKAGC |
| | (SEQ ID NO: 150) |
| Hu_aTNFa | QVQLVESGGGLVQPGGSLRLSCAASGRTFSDHSGYTYTMGWFRQAPG |
| Hu_7MUT | KEREFVARIYWSSGNTYYADSVKGRFTISRDNSKNTVYLQMNSLRPED |
| aH_CYS | TAVYYCAARDGIPTSRSVESYNYWGQGTLVTVSSAEAAAKEAAAKEA |
| | AAKAGC (SEQ ID NO: 151) |
| IL- | APTSSSTKKTQLQLEHLLLDLQMILNGINNYKNPKLTRMLTFKFYMPKK |
| 2_C125S | ATELKHLQCLEEELKPLEEVLNLAQSKNFHLRPRDLISNINVIVLELKGS |
| aH_CYS | ETTFMCEYADETATIVEFLNRWITFSQSIISTLTAEAAAKEAAAKEAAAK |
| | AGC (SEQ ID NO: 152) |
| IL- | NWVNVISDLKKIEDLIQSMHIDATLYTESDVHPSCKVTAMQCFLSELQV |
| 15_5MUT | ISLESGDASIHDTVENLTILANNSLSSNGYVTESGCKECEELEAKNIKEFL |
| aH_CYS | QSFVHIVQMFINTSAEAAAKEAAAKEAAAKAGC (SEQ ID NO: 153) |
| aTNFa_ | DLGKKLLEVARAGQDDEVRILMANGADVNAADHQSFTPLHLYAIFGH |
| DARPin | LEIVEVLLKNGADVNASDWHGNTPLHLAAWIGHLEIVEVLLKYGADV |
| G3S_CYS | NATDHSGSTPLHLAATLGHLEIVEVLLKYGADVNAQDKFGKTAFDISID |
| | NGNEDLAEILQKAAGGGSGGGSC (SEQ ID NO: 154) |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirely to the same extent as if each reference was individually incorporated by reference. Where a conflict exists between the instant application and a reference provided herein, the instant application shall dominate.

What is claimed is:

1. A method for treating uveitis in a subject in need thereof, comprising administering to the subject a therapeutically effective amount of a conjugate of Formula V:
(X—Y)ₙ—Z (V),
wherein
each X is independently an anti-inflammatory peptide having a molecular weight of from about 5 kDa to about 200 kDa;
each Y is an organic linker;
Z is a hyaluronic acid polymer having a molecular weight of from about 0.1 MDa to about 3 MDa; and
subscript n is an integer of from 1 to 1000.

2. The method of claim 1, wherein each X is independently an anti-TNF-α peptide or an anti-interleukin-1β peptide.

3. The method of claim 1 or 2, wherein each X is a monoclonal IgG, an IgG fragment, single chain scFv, single-domain heavy-chain VHH, adnectin, affibody, anticalin, DARPin, or an engineered Kunitz-type inhibitor.

4. The method of any one of claims 1 to 3, wherein
each X is a peptide having an amino acid sequence comprising any one of SEQ ID NOS: 61-73, 81-85, 91-98, 101-109, 111-118, and 145-154.

5. The method of any one of claims 1 to 3, wherein
each X is a peptide having an amino acid sequence comprising:
(SEQ ID NO: 101)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSPSTPPTPS PSTPPGGCDD
DDK,
(SEQ ID NO: 102)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSAEAAAKEA AAKEAAAKAG
C,
(SEQ ID NO: 103)
QVQLQDS GGGLVQAGGS LRLSCAASGG TFSSIIMAWF RQAPGKEREF
VGAVSWSGGT TVYADSVLGR FEISRDSARK SVYLQMNSLK PEDTAVYYCA
ARPYQKYNWA SASYNVWGQG TQVTVSSAEA AAKEAAAKEA AAKAGC,
(SEQ ID NO: 104)
QVQLQES GGGLVQAGGS LRLSCAASGG TFSSIIMAWF RQAPGKEREF
VGAVSWSGGT TVYADSVKGR FTISRDSARK SVYLQMNSLK PEDTAVYYCA
ARPYQKYNWA SASYNVWGQG TQVTVSSAEA AAKEAAAKEA AAKAGC,
(SEQ ID NO: 105)
CGGGVDNKFN KEVGWAFGEI GALPNLNALQ FRAFIISLWD DPSQSANLLA
EAKKLNDAQA PK,
or
(SEQ ID NO: 106)
EIVMTQS PSTLSASVGD RVIITCQASQ SIDNWLSWYQ QKPGKAPKLL
IYRASTLASG VPSRFSGSGS GAEFTLTISS LQPDDFATYY CQNTGGGVSI
AFGQGTKLTV LGGGGGSGGG GSGGGGSGGG GSEVQLVESG GGLVQPGGSL
RLSCTASGFS LSSAAMAWVR QAPGKGLEWV GIIYDSASTY YASWAKGRFT
ISRDTSKNTV YLQMNSLRAE DTAVYYCARE RAIFSGDFVL WGQGTLVTVS
SSPSTPPTPS PSTPPGGC.

6. The method of any one of claims 1 to 5, wherein
each Y is an organic linker having the structure: 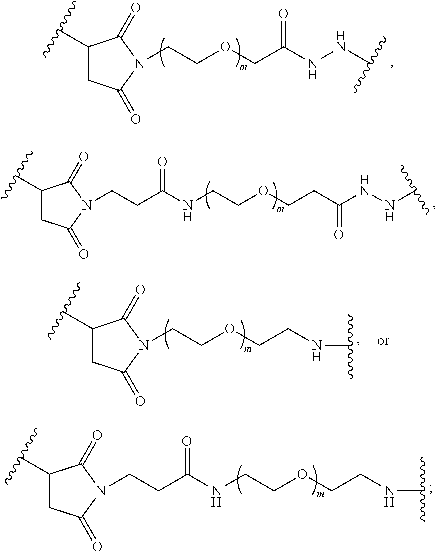
and
subscript m is an integer of from 1 to 300.

7. The method of any one of claims 1 to 5, wherein Z has a molecular weight of from about 0.4 MDa to about 2 MDa.

8. The method of any one of claims 1 to 7, wherein Z has a molecular weight of from about 0.7 MDa to about 1.5 MDa.

9. The method of any one of claims 1 to 8, wherein Z has a molecular weight of about 0.8 MDa.

10. The method of any one of claims 1 to 9, wherein conjugate of Formula V has the structure of Formula Va:
(X¹—X²—Y)ₙ—Z (Va),
wherein
each X¹ is an anti-inflammatory peptide having a molecular weight of from about 5 kDa to about 200 kDa;
each X² is a peptide linker that comprises an alpha-helix;
each Y is an organic linker having the structure: 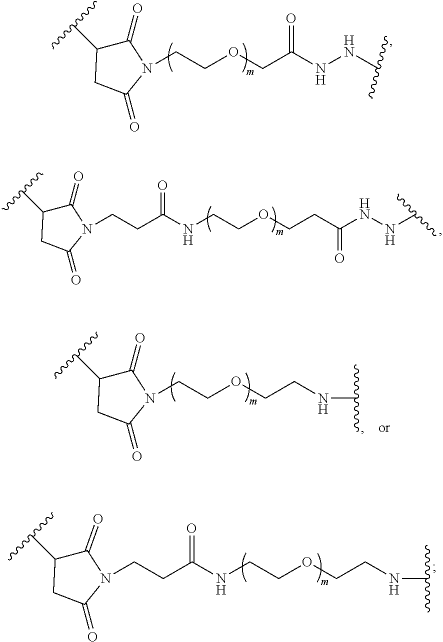
Z is a hyaluronic acid polymer having a molecular weight of from about 0.1 MDa to about 3 MDa; and
subscript m is an integer of from 1 to 300.

11. The method of claim 10, wherein
each X² is a peptide linker having an amino acid sequence comprising:
(SEQ ID NO: 21)
AEAAAKEAAAKEAAAKAGC,
(SEQ ID NO: 22)
AEEEKRKAEEEKRKAEEEAGC,
(SEQ ID NO: 23)
AEEEKRKAEEEKRKAEEEKRKAEEEAGC,
(SEQ ID NO: 24)
AEEEEKKKKEEEEKKKKAGC,
(SEQ ID NO: 25)
AEAAAKEAAAKAGC,
(SEQ ID NO: 26)
PSRLEEELRRRLTEGC,
or
(SEQ ID NO: 27)
AEEEEKKKQQEEEAERLRRIQEEMEKERKRREEDEERRRKEEEER
RMKLEMEAKRKQEEEERKKREDDEKRKKKAGC.

12. The method of any one of claims 1 to 11, wherein the conjugate of Formula V is a random polymer of Formula VI:
(X—Y—Z¹)ₙ—(Z²)ₚ—(Z³)_{q} (VI),
having a molecular weight of from about 0.1 MDa to about 3 MDa;
wherein
each X is independently an anti-inflammatory peptide having a molecular weight of from about 5 kDa to about 200 kDa;
each Y is an organic linker;
each X—Y—Z¹ moiety has the structure: 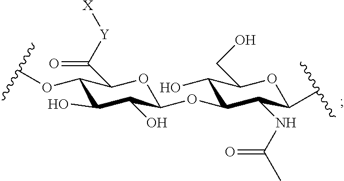
each Z² has the structure: 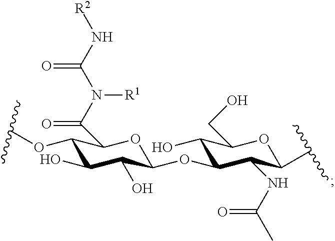
each Z³ independently has the structure: 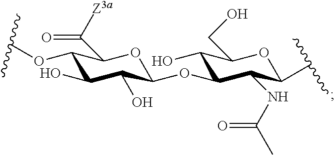
each R¹ and R² is independently C₁-C₆ alkyl, —(C₁-C₆ alkyl)-NR³R⁴, or C₅-C₈ cycloalkyl;
each R³ and R⁴ is independently H or C₁-C₆ alkyl;
each Z³a is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

13. The method of claim 12, wherein each R¹ and R² is independently C₁-C₃ alkyl or —(C₁-C₃ alkyl)-NR³R⁴.

14. The method of claim 12 or 13, wherein each R³ and R⁴ is independently C₁-C₃ alkyl.

15. The method of any one of claims 12 to 14, wherein
subscript n is an integer of from 1 to 1500 and less than about 15% of the sum of subscripts n, p, and q;
subscript p is an integer of from 1 to 1000 and less than about 10% of the sum of subscripts n, p, and q; and
subscript q is an integer of from 100 to 10000.

16. The method of any one of claims 12 to 15, wherein
subscript n is an integer of from 1 to 1000 and less than about 10% of the sum of subscripts n, p, and q;
subscript p is an integer of from 1 to 800 and less than about 8% of the sum of subscripts n, p, and q; and
subscript q is an integer of from 100 to 10000.

17. The method of any one of claims 12 to 16, wherein
subscript n is an integer of from 10 to 450 and less than about 15% of the sum of subscripts n, p, and q;
subscript p is an integer of from 1 to 300 and less than about 10% of the sum of subscripts n, p, and q; and
subscript q is an integer of from 1000 to 3000.

18. The method of any one of claims 12 to 17, wherein
subscript n is an integer of from 10 to 300 and less than about 10% of the sum of subscripts n, p, and q;
subscript p is an integer of from 1 to 240 and less than about 8% of the sum of subscripts n, p, and q; and
subscript q is an integer of from 1000 to 3000.

19. The method of any one of claims 12 to 18, wherein
subscript n is an integer of from 10 to 300 and less than about 10% of the sum of subscripts n, p, and q;
subscript p is an integer of from 1 to 60 and less than about 2% of the sum of subscripts n, p, and q; and
subscript q is an integer of from 1000 to 3000.

20. The method of any one of claims 12 to 19, wherein
subscript n is an integer of from 10 to 300 and less than about 10% of the sum of subscripts n, p, and q;
subscript p is an integer of from 1 to 30 and less than about 1% of the sum of subscripts n, p, and q; and
subscript q is an integer of from 1000 to 3000.

21. The method of any one of claims 12 to 20, wherein
subscript n is an integer of from 10 to 300 and less than about 10% of the sum of subscripts n, p, and q;
subscript p is an integer of from 1 to 15 and less than about 0.5% of the sum of subscripts n, p, and q; and
subscript q is an integer of from 1000 to 3000.

22. The method of any one of claims 1 to 21, wherein the uveitis is chronic uveitis.

23. The method of any one of claims 1 to 22, wherein the uveitis is chronic non-infectious uveitis.

24. The method of any one of claims 1 to 23, comprising intravitreal administration.

25. The method of any one of claims 1 to 24, comprising multiple administrations of the conjugate.

26. The method of claim 25, comprising administering the conjugate every month, every two months, or every three months.

27. The method of claim 25, comprising administering the conjugate twice or three times yearly.

28. The method of claim 25, comprising administering the conjugate yearly.

29. A conjugate that is a random polymer of Formula VI:
(X—Y—Z¹)ₙ—(Z²)ₚ—(Z³)_{q} (VI),
having a molecular weight of from about 0.1 MDa to about 3 MDa;
wherein
each X is independently an anti-TNF-α or anti-IL-1β peptide comprising:
(SEQ ID NO: 101)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSPSTPPTPS PSTPPGGCDD
DDK,
(SEQ ID NO: 102)
QVQLQES GGGLVQPGGS LRLSCAASGR TFSDHSGYTY TIGWFRQAPG
KEREFVARIY WSSGNTYYAD SVKGRFAISR DIAKNTVDLT MNNLEPEDTA
VYYCAARDGI PTSRSVESYN YWGQGTQVTV SSAEAAAKEA AAKEAAAKAG
C,
(SEQ ID NO: 103)
QVQLQDS GGGLVQAGGS LRLSCAASGG TFSSIIMAWF RQAPGKEREF
VGAVSWSGGT TVYADSVLGR FEISRDSARK SVYLQMNSLK PEDTAVYYCA
ARPYQKYNWA SASYNVWGQG TQVTVSSAEA AAKEAAAKEA AAKAGC,
(SEQ ID NO: 104)
QVQLQES GGGLVQAGGS LRLSCAASGG TFSSIIMAWF RQAPGKEREF
VGAVSWSGGT TVYADSVKGR FTISRDSARK SVYLQMNSLK PEDTAVYYCA
ARPYQKYNWA SASYNVWGQG TQVTVSSAEA AAKEAAAKEA AAKAGC,
(SEQ ID NO: 105)
CGGGVDNKFN KEVGWAFGEI GALPNLNALQ FRAFIISLWD DPSQSANLLA
EAKKLNDAQA PK,
or
(SEQ ID NO: 106)
EIVMTQS PSTLSASVGD RVIITCQASQ SIDNWLSWYQ QKPGKAPKLL
IYRASTLASG VPSRFSGSGS GAEFTLTISS LQPDDFATYY CQNTGGGVSI
AFGQGTKLTV LGGGGGSGGG GSGGGGSGGG GSEVQLVESG GGLVQPGGSL
RLSCTASGFS LSSAAMAWVR QAPGKGLEWV GIIYDSASTY YASWAKGRFT
ISRDTSKNTV YLQMNSLRAE DTAVYYCARE RAIFSGDFVL WGQGTLVTVS
SSPSTPPTPS PSTPPGGC;
each Y is an organic linker having the structure: 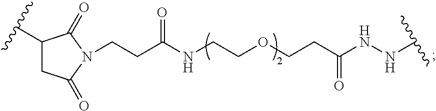
each X—Y—Z¹ moiety has the structure: 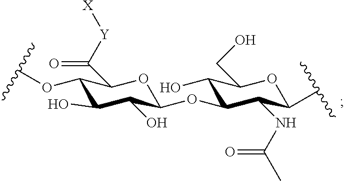
each Z² has the structure: 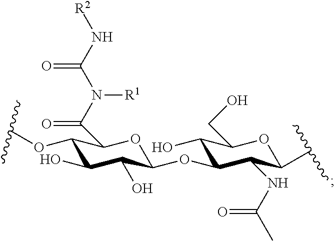
each Z³ independently has the structure: 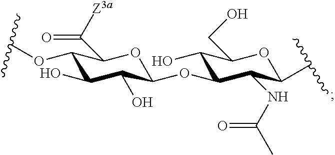
each R¹ and R² is independently C₁-C₆ alkyl, —(C₁-C₆ alkyl)-NR³R⁴, or C₅-C₈ cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.
